SEQUENCE LISTING (18 sequences)

SEQ ID NO: 1 - protein, 65 aa, synthetic construct
TRIVVSGRGDIPLQLVREVAKLARELGARVTVHGRIVTIEVDTEEEERAVRELAEELGLFYYTEK

SEQ ID NO: 2 - protein, 65 aa, synthetic construct
LELEISGRGDVPTQFIQKLAKRLKELGLEVTNRYGVVKVRGIDEELAERIKKEAEEHGIFVSFSG

SEQ ID NO: 3 - protein, 65 aa, synthetic construct
GEVEVSGRGDIPRRSLELFEKVAKELGLKVERNGYQVTVKGVSEEQIRELEEVAKKLGLFVRVTE

SEQ ID NO: 4 - protein, 65 aa, synthetic construct
TTVFVSGRGDFPTRILEELEELAKKLGLEVHKNGRQLEVRDDDEEALRRFAEIARELGAFVIVRH

SEQ ID NO: 5 - protein, 65 aa, synthetic construct
TELIINGRGDFPSSELERLRERAERLGIKVRVRLGVLHLKGISEEEAERLARELRKRGIFVEIRK

SEQ ID NO: 6 - protein, 65 aa, synthetic construct
IEIVISGRGDFPLSELLRLAEELRRLGAEVHLRLGVIRIHVDDEELAREIEERAKKAGAFVIRHD

SEQ ID NO: 7 - protein, 65 aa, synthetic construct
TTIDVNGRGDLPQRLAREFAEWAKRLGLEVTLHGRRVTVRLDDEELAREVEEYAKKLGLFVYVHG

SEQ ID NO: 8 - protein, 65 aa, synthetic construct
SEIEVSGRGDFPSQVLYRLAEIARELGLEVHLRGLTLHIRGDDEEAVKEILRIIKELGLFVIVRE

SEQ ID NO: 9 - protein, 65 aa, synthetic construct
LRIRISGRGDVPTQILERLEKRARELGLTVRLNGLQVTIEGISEEEFRELREEAERNGLFVERLE

SEQ ID NO: 10 - protein, 71 aa, synthetic construct
GELKVELRGDTDTRLVRRLLEEIEKKYPEVRVESRNWGREQTVTGLDEEAIKRLAEELKRKFQFLTIRRTE

SEQ ID NO: 11 - protein, 65 aa, synthetic construct
TELVIHGRGDFPSSEMAKLKKELEEKGIEVEVKHKVLTAKGISKEEAERLAKELESRGVWTEIKE

SEQ ID NO: 12 - protein, 65 aa, synthetic construct
SELIIHGRGDFPSSELERLRERFERLGIKVRVDHKVLTLIGISEEEAERLARELRKRGIWVEIRK

SEQ ID NO: 13 - protein, 67 aa, synthetic construct
GEVEVHGRGDIPRSSLELFEKVAKELGLKVERNHRTVTVKGVSEEQIRELEEVAKKLGLWVLVRVTE

SEQ ID NO: 14 - protein, 65 aa, synthetic construct
SELIIHGRGDFPRSELERLRERFERLGIKVRVDHKVLTLIGISEEEAERLARELRKRGIWVEIRK

SEQ ID NO: 15 - protein, 6 aa, synthetic construct
GRGDSP

SEQ ID NO: 16 - protein, 163 aa, synthetic construct
  VARIANT 1: note = optionally absent
  VARIANT 148..163: note = optionally absent
MSGRQEKVLKSIEETVRKMGVTMETHRSGNEVKVVIKGLHIKQQRQLYRDVRETSKKQGVETEIEVEGDTVTIVVREGGSGGSELIIHGRGDFPRSELERLRERFERLGIKVRVDHKVLTLIGISEEEAERLARELRKRGIWVEIRKGSGSHHWGSTHHHHHH

SEQ ID NO: 17 - protein, 289 aa, synthetic construct
  VARIANT 1: note = optionally absent
  VARIANT 282..289: note = optionally absent
MGRQEKVLKSIEETVRKMGVTMETHRSGNEVKVVIKGLHESQQEQLLEDVLRTAEKQGVRVRIRFKGDTVTIVVREGSPEEILERAKESLERAKEAFERGDEEEFRKAAEKALELAKRLVEQAKKEGDPELVFEAARVALWVAWLAAWFGDKEVFKKAAESALEVAKRLVEVAKEEGDPELVLKAAFVALLVAIMAVILGDKEVFKKAAESALEVAKRLVEIAAREGDPELVEEAAKVAELVRELAKLMGDEEVYEKARETAREVRLFLLFVRIWEGGGGSLEHHHHHH

SEQ ID NO: 18 - protein, 77 aa, synthetic construct
  VARIANT 1: note = optionally absent
MSGRQEKVLKSIEETVRKMGVTMETHRSGNEVKVVIKGLHIKQQRQLYRDVRETSKKQGVETEIEVEGDTVTIVVRE

(19) United States
(12) Patent Application Publication
(10) Pub. No.: US 2024/0383964 A1
(43) Pub. Date: Nov. 21, 2024

(54) De Novo Designed alpha(5) beta(1) Integrin selective minibinders

(71) Applicant: UNIVERSITY OF WASHINGTON, Seattle, WA (US)

(72) Inventors: David BAKER, Seattle, WA (US); Xinru WANG, Seattle, WA (US); Jordi GUILLEM MARTI, Seattle, WA (US); Buwei HUANG, Seattle, WA (US)

(21) Appl. No.: 18/662,616

(22) Filed: May 13, 2024

Related U.S. Application Data
(60) Provisional application No. 63/570,567, filed on Mar. 27, 2024.
(60) Provisional application No. 63/502,292, filed on May 15, 2023.

(51) Int. Cl. C07K 14/705; A61K 38/00; A61K 45/06; A61P 9/00

(52) U.S. Cl. CPC C07K 14/7055; A61K 45/06; A61P 9/00; A61K 38/00; C07K 2319/70

(57) ABSTRACT

Integrin α5β1-binding polypeptides are disclosed having an amino acid sequence at least 50% identical to the amino acid sequence selected from SEQ ID NO:1-14, or selected from SEQ ID NO:11-14, not including any insertions, wherein residues 8-10 relative to the reference polypeptide are RGD, and their use for treating cancer, vascular disease, rheumatoid arthritis, and diseases associated with pathogenic angiogenesis.

### FEDERAL FUNDING STATEMENT

This invention was made with government support under Grant No. R01AG063845-01, awarded by the National Institute on Aging. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on May 13, 2024 having the file name “23-0228-US.xml” and is 19,769 bytes in size.

### BACKGROUND

Integrins are a type of cell adhesion molecule that play a critical role in various biological processes, including cell migration, differentiation, and survival. The integrin alpha (5) beta (1) (“α5β1”) has been implicated in the development and progression of cancer and other diseases. However, designing integrin α5β1 binders has been challenging because of the high conservation of the RGD-binding integrin members.

### SUMMARY

In one aspect, the disclosure provides polypeptides comprising or consisting of an amino acid sequence at least 50%, 55%, 60%, 70%, 75%, 80%, 85%, 90%, 91%, 92%, 93%, 94%, 95%, 96%, 97%, 98%, 99%, or 100% identical to the amino acid sequence selected from SEQ ID NO:1-14, not including any insertions, wherein residues 8-10 relative to the reference polypeptide are RGD, and wherein the polypeptide selectively binds to α(5)β(1) integrin. In one embodiment, the polypeptides comprise or consist of an amino acid sequence at least 50%, 55%, 60%, 70%, 75%, 80%, 85%, 90%, 91%, 92%, 93%, 94%, 95%, 96%, 97%, 98%, 99%, or 100% identical to the amino acid sequence selected from SEQ ID NO:11-14, not including any insertions, wherein the polypeptide selectively binds to α(5)β(1) integrin.

In some embodiments, at least 1, 2, 3, 4, 5, 6, 7, 8, 9, 10, 11, 12, 13, 14, 15, or all 16 interface residues in addition to residues 8-10 are identical to those in the reference polypeptide. In other embodiments, at least 1, 2, 3, 4, 5, 6, 7, or all 8 core residues are identical to those in the reference polypeptide. In further embodiments, substitutions relative to the reference polypeptide are selected from those listed in Table 2, Option 1. In other embodiments, substitutions relative to the reference polypeptide are selected from those listed in Table 2, Option 2. In some embodiments, substitutions relative to the reference polypeptide are conservative amino acid substitutions.

In various embodiments, relative to the reference polypeptide, 1, 2, 3, 4, 5, 6, 7, 8, 9, 10, 11, 12, or all 13 of the following are true:
Residue 3 is L or V;
Residue 5 is I or V;
Residue 6 is H;
Residue 13 is S or R;
Residue 14 is S;
Residue 31 is E or R;
Residue 32 is V or R;
Residue 33 is D, K, or N;
Residue 34 is H;
Residue 35 is K or R;
Residue 58 is G;
Residue 59 is I, V, or L; and/or
Residue 60 is W.

In other embodiments, relative to the reference polypeptide, 1, 2, 3, 4, 5, 6, 7, or all 8 of the following are true:
Residue 31 is E or R;
Residue 32 is V or R;
Residue 33 is D, K, or N;
Residue 34 is H;
Residue 35 is K or R;
Residue 58 is G;
Residue 59 is I, V, or L; and/or
Residue 60 is W.

In one embodiment, relative to the reference polypeptide, residue 12 is P. In another embodiment, substitutions relative to the reference polypeptide are selected from those listed in Table 2, Option 3. In one embodiment, the polypeptides comprise an insertion in one or more loop regions of the polypeptide.

In another embodiment, the disclosure provides fusion proteins, comprising a polypeptide of any embodiment or combination of embodiments herein, and one or more functional domains. In one embodiment, the one or more functional domains comprises a multimerization domain.

In another embodiment, the polypeptide or fusion protein of any embodiment or combination of embodiments herein, binds integrin α5β1 with an affinity of between about 0.025 nm and about 50 μm, or between about 0.05 nm and about 8 μm, or between about 0.05 nm and about 8 μm, or between about 0.05 nm and about 2.5 μm, or between about 0.05 nm and about 700 nm, or between about 0.05 nm and about 0.5 nm.

In other embodiments, the disclosure provides nucleic acids encoding the polypeptide or fusion protein of any embodiment or combination of embodiments herein, expression vectors comprising the nucleic acid operatively linked to a suitable control sequence (such as a promoter), host cells comprising the polypeptide, fusion protein, nucleic acid, or expression vector of any embodiment or combination of embodiments herein, and pharmaceutical compositions comprising the polypeptide, the nucleic acid, the expression vector, and/or the host cell of any embodiment or combination of embodiments herein, and a pharmaceutically acceptable carrier.

In another aspect, the disclosure provides methods for treating cancer, comprising administering to a subject having cancer an amount effective to treat the cancer of the polypeptide, fusion protein, nucleic acid, expression vector, host cell, and/or pharmaceutical composition of any embodiment or combination of embodiments herein.

In a further aspect, the disclosure provides methods for inhibiting tumor metastasis, comprising administering to a subject having cancer an amount effective to inhibit tumor metastasis of the polypeptide, fusion protein, nucleic acid, expression vector, host cell, and/or pharmaceutical composition of any embodiment or combination of embodiments herein.

The disclosure also provides methods for treating cancer, comprising administering to a subject undergoing radiation therapy to treat cancer an amount of the polypeptide, fusion protein, nucleic acid, expression vector, host cell, and/or pharmaceutical composition of any embodiment or combination of embodiments herein effective to enhance effectiveness of the radiation therapy.

The disclosure further provides methods for inhibiting cell migration and/or attachment, comprising administering to a subject in need thereof an amount effective to inhibit cell migration and/or attachment of the polypeptide, fusion protein, nucleic acid, expression vector, host cell, and/or pharmaceutical composition of any embodiment or combination of embodiments herein.

The disclosure also provides methods for treating a vascular disease or rheumatoid arthritis, comprising administering to a subject having a vascular disease or rheumatoid arthritis an amount effective to treat the vascular disease or rheumatoid arthritis of the polypeptide, fusion protein, nucleic acid, expression vector, host cell, and/or pharmaceutical composition of any embodiment or combination of embodiments herein.

In another aspect, the disclosure provides medical devices or implants comprising a plurality of the polypeptide, fusion protein, or pharmaceutical composition of any embodiment or combination of embodiments herein coated on or in the medical device or implant.

### DESCRIPTION OF THE FIGURES

FIG. 1. (A) Illustration of integrin conformations. (B) RGD binding integrins. (C) Integrin α5β1 (PDB:4WK2) and αvβ3 (PDB:4G1M) RGD binding site overlay. (D) Electrostatic representation of the head piece of integrin α5β1, αvβ3, αvβ6, and αvβ8. (E) Computational design pipeline of integrin specific binders. RGD containing protein binders were designed by either building RGD sequence in ferredoxin scaffolds or grafted on previously build ferredoxin scaffolds. Designs were sequenced, designed, and filtered with the aid of Rosetta™.

FIG. 2. (A) BLI binding affinity traces for minibinder or ⁸RGD¹⁰/GGG mutant against α5β1 in indicated buffers. Minibinder affinity to integrin α5β1 is dependent on metal ion presence and RGD motif. All affinity data was collected by Octet™ R8 and binding affinity was estimated by the Octet™ ForteBio software package. (B) Minibinder has no detectable affinity in presence of 500 nM or 1000 nM integrin αvβ1, αvβ3, αvβ6, αvβ8 and α8β1. (C) Cell surface titration of minibinder 4584_V5.3 and 00179_v4.1 against K562 cells wild type. (D) Minibinder 00179_v4.1 is conjugated to GFP-tagged protein sheets and colocalized with active form of integrin α5β1. Integrin α5 subunit and β1 subunit were stained with integrin α5 and active integrin β1 subunit specific antibody, respectively. (E) Quantification of (D). Quantification was counted as a percentage of mb-sheets that colocalized with staining of a5 and b1, a5 only, b1 only, or none; 30-35 sheets were counted in each biological repeat. (F) Representative decrease in vascular stability by minibinder 00179_v4.1. Minibinders were added to HUVEC cells at 0, 0.1, 1, 10, and 100 nM. Vascular stability was analyzed at 12-h timepoints. 20 images were taken in each well at random locations. The scale bar is 100 μm. (G) Quantification of (F). The number of nodes, meshes, and tubes from images in (F) were quantified using an angiogenesis analyzer plug-in in ImageJ. Statistical significance of each were analyzed using One-way Anova Bonferroni's multiple comparison test. (H) Minibinder alone or C6-oligomer inhibits cell migration relative to PBS/H8 control. The wound area of T0 and T12 time points were quantified using imageJ and level of cell migration were calculated using this equation (AreaT0-T12)/AreaT0, all samples were normalized to PBS as fold change.

FIG. 3. Site saturation mutagenesis (SSM analysis) of exemplary primary hits. RGD motifs are highlighted.

FIG. 4. High affinity binder variants were identified through combo libraries screening using yeast-display assay. Yeast libraries expressing integrin α5β1 binder variants encoding beneficial mutations identified in SSM analysis were incubated with 200 pico-molar biotinylated integrin α5β1 for an hour. Then the yeasts were washed, and further stained with both streptavidin-phycoerythrin (SAPE, ThermoFisher) and anti-c-Myc fluorescein isothiocyanate (FITC, Miltenyi Biotech) at 1:100 ratio for 30 min. After washing twice with buffer, the yeast cells bound to integrin α5β1 were sorted via FACS.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), RosettaCommons.org, and the Ambion 1998 Catalog (Ambion, Austin, TX).

As used herein, the singular forms “a”, “an” and “the” include plural referents unless the context clearly dictates otherwise.

As used herein, “about” means +/−5% of the recited value.

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Any N-terminal methionine residue in any polypeptide of the disclosure may be present or may be deleted.

In a first aspect, the disclosure provides polypeptides comprising or consisting of an amino acid sequence at least 50%, 55%, 60%, 70%, 75%, 80%, 85%, 90%, 91%, 92%, 93%, 94%, 95%, 96%, 97%, 98%, 99%, or 100% identical to the amino acid sequence selected from SEQ ID NO:1-14, not including any insertions (i.e., any insertions are not considered when determining percent identity to the reference polypeptide), wherein residues 8-10 relative to the reference polypeptide are RGD, and wherein the polypeptide selectively binds to integrin α5β1.

The polypeptides of the disclosure selectively bind to α5β1 integrin with antagonistic activity. The term “antagonistic activity” is used herein to describe the activity of a polypeptide that, upon binding to α5β1 integrin, inactivates the integrin complex or inhibits processes normally initiated by the integrin. In some embodiments, the polypeptide or fusion protein of any embodiment or combination of embodiments herein, binds integrin α5β1 with an affinity of between about 0.025 nm and about 50 μm, or between about 0.05 nm and about 8 μm, or between about 0.05 nm and about 8 μm, or between about 0.05 nm and about 2.5 μm, or between about 0.05 nm and about 700 nm, or between about 0.05 nm and about 0.5 nm. The binding affinity can be measured, for example, using biolayer interferometry (BLI) or yeast display. The polypeptides bind selectively to integrin α5β1, in that no detectable binding was observed in the presence of 500 nM of integrin a8b1, avb1, avb6, avb8, and avb3 using biolayer interferometry (BLI) in integrin assay buffer (20 mM Tris pH 7.4, 150 mM NaCl, 1 mM MgCl₂, 1 mM CaCl₂, 0.02% tween-20), as described in the examples. In one such assay to measure the binding affinity to a51integrin, the polypeptides are cloned with an N-terminus avi-tag and biotinylated chemically. Prior to measurements, streptavidin-coated biosensors are first hydrated in assay buffer for 10 minutes. Biotinylated polypeptides are immobilized onto the biosensors by dipping them into a solution with 100 nM protein until the response reached between 10% and 50% of the maximum value followed by dipping sensors into fresh buffer to establish a baseline for 120 s. Association of integrins is allowed by dipping biosensors in solutions containing designed protein diluted in assay buffer until equilibrium is approached followed by dissociation by dipping the biosensors into fresh buffer solution to monitor the dissociation kinetics. The association and dissociation with all the different binders is allowed for 900-1500 s for each step. Global kinetic or steady-state fits are performed on buffer-subtracted data assuming a 1:1 binding model.

The amino acid sequences of SEQ ID NO:1-14 are shown in Table 1, and bound integrin α5β1 with an affinity of between about 0.05 nm and about 50 μm, with the designs of SEQ ID NO:11-13 having an affinity of between about 0.05 nm and about 0.5 nm.

TABLE 1

| SEQ | | |
|---|---|---|
| ID | | |
| NO | Name | Amino acid sequence |
| 1 | xw_a5b1_ | TRIVVSGRGDIPLQLVREVAKLARE |
| | 04086 | LGARVTVHGRIVTIEVDTEEEERAV |
| | | RELAEELGLFYYTEK |
| 2 | xw_a5b1_ | LELEISGRGDVPTQFIQKLAKRLKE |
| | 20658 | LGLEVTNRYGVVKVRGIDEELAERI |
| | | KKEAEEHGIFVSFSG |
| 3 | xw_a5b1_ | GEVEVSGRGDIPRRSLELFEKVAKE |
| | 04584 | LGLKVERNGYQVTVKGVSEEQIREL |
| | | EEVAKKLGLFVRVTE |
| 4 | xw_a5b1_ | TTVFVSGRGDFPTRILEELEELAKK |
| | 04545 | LGLEVHKNGRQLEVRDDDEEALRRF |
| | | AEIARELGAFVIVRH |
| 5 | xw_a5b1_ | TELIINGRGDFPSSELERLRERAER |
| | 00179 | LGIKVRVRLGVLHLKGISEEEAERL |
| | | ARELRKRGIFVEIRK |
| 6 | xw_a5b1_ | IEIVISGRGDFPLSELLRLAEELRR |
| | 03886 | LGAEVHLRLGVIRIHVDDEELAREI |
| | | EERAKKAGAFVIRHD |
| 7 | xw_a5b1_ | TTIDVNGRGDLPQRLAREFAEWAKR |
| | 01280 | LGLEVTLHGRRVTVRLDDEELAREV |
| | | EEYAKKLGLFVYVHG |
| 8 | xw_a5b1_ | SEIEVSGRGDFPSQVLYRLAEIARE |
| | 21894 | LGLEVHLRGLTLHIRGDDEEAVKEI |
| | | LRIIKELGLFVIVRE |
| 9 | xw_a5b1_ | LRIRISGRGDVPTQILERLEKRARE |
| | 20232 | LGLTVRLNGLQVTIEGISEEEFREL |
| | | REEAERNGLFVERLE |
| 10 | JD_a5b1_ | GELKVELRGDTDTRLVRRLLEEIEK |
| | 3313 | KYPEVRVESRNWGREQTVTGLDEEA |
| | | IKRLAEELKRKFQFLTIRRTE |
| 11 | xw_a5b1_ | TELVIHGRGDFPSSEMAKLKKELEE |
| | 00179_v | KGIEVEVKHKVLTAKGISKEEAERL |
| | 4.1_mpnn6 | AKELESRGVWTEIKE |
| 12 | xw_a5b1_ | SELIIHGRGDFPSSELERLRERFER |
| | 00179_v | LGIKVRVDHKVLTLIGISEEEAERL |
| | 4.1 | ARELRKRGIWVEIRK |
| 13 | xw_a5b1_ | GEVEVHGRGDIPRSSLELFEKVAKE |
| | 004584 | LGLKVERNHRTVTVKGVSEEQIREL |
| | v5.3 | EEVAKKLGLWVLVRVTE |
| 14 | xw_a5b1_ | SELIIHGRGDFPRSELERLRERFER |
| | 00179_ | LGIKVRVDHKVLTLIGISEEEAERL |
| | v4.1 | ARELRKRGIWVEIRK |
| | point | |
| | mutant | |
| | used in | |
| | hexamer | |

The position of residues in the polypeptides of the disclosure are “relative to” the position of residues in the reference sequence: this does not necessarily mean that the residue number in the polypeptide of the disclosure will be identical to the residue number in the reference sequence. For example, residues 8-10 of the reference sequences of SEQ ID NO:1-14 are RGD. Polypeptides of the disclosure have an RGD tripeptide that is at a position corresponding to residues 8-10 in the reference sequence, but not necessarily at residues 8-10 in the polypeptides of the disclosure. Those of skill in the art will understand that the polypeptides of the disclosure may be fused to other functional domains (such as the fusion proteins or polypeptides with insertions described below), including N-terminal domains, or comprise insertions, such that the RGD residues in the polypeptides will not be at positions 8-10, but the polypeptides will still have the RGD triad at residues 8-10 relative to the reference polypeptide selected from SEQ ID NO:1-14.

In some embodiments, the polypeptides comprise an amino acid sequence at least 50%, 55%, 60%, 70%, 75%, 80%, 85%, 90%, 91%, 92%, 93%, 94%, 95%, 96%, 97%, 98%, 99%, or 100% identical to the amino acid sequence selected from SEQ ID NO:11-14, not including any insertions, wherein the polypeptide selectively binds to integrin α5β1. In another embodiment, the polypeptides consist of an amino acid sequence at least 50%, 55%, 60%, 70%, 75%, 80%, 85%, 90%, 91%, 92%, 93%, 94%, 95%, 96%, 97%, 98%, 99%, or 100% identical to the amino acid sequence selected from SEQ ID NO:11-14, not including any insertions, wherein the polypeptide selectively binds to integrin α5β1.

In various embodiments, the polypeptides comprise an amino acid sequence at least 60% identical to the amino acid sequence selected from SEQ ID NO:1-14 or 11-14; or at least 70% identical to the amino acid sequence selected from SEQ ID NO:1-14 or 11-14; or at least 80% identical to the amino acid sequence selected from SEQ ID NO:1-14 or 11-14; or at least 90% identical to the amino acid sequence selected from SEQ ID NO:1-14 or 11-14; at least 95% identical to the amino acid sequence selected from SEQ ID NO:1-14 or 11-14; in all embodiments not including any insertions, wherein residues 8-10 relative to the reference polypeptide are RGD, and wherein the polypeptide selectively binds to integrin α5β1. In other embodiments, the polypeptides consist of an amino acid sequence at least 60% identical to the amino acid sequence selected from SEQ ID NO:1-14 or 11-14; or at least 70% identical to the amino acid sequence selected from SEQ ID NO:1-14 or 11-14; or at least 80% identical to the amino acid sequence selected from SEQ ID NO:1-14 or 11-14; or at least 90% identical to the amino acid sequence selected from SEQ ID NO:1-14 or 11-14; at least 95% identical to the amino acid sequence selected from SEQ ID NO:1-14 or 11-14; in all embodiments not including any insertions, wherein residues 8-10 relative to the reference polypeptide are RGD, and wherein the polypeptide selectively binds to integrin α5β1.

In further embodiments, at least 1, 2, 3, 4, 5, 6, 7, 8, 9, 10, 11, 12, 13, 14, 15, or all 16 interface residues in addition to residues 8-10 are identical to those in the reference polypeptide. The interface residues are those at the interface between the reference polypeptide and integrin α5β1.

Table 2 shows the position of interface residues in the polypeptides, including RGD residues at positions 8-10 relative to the reference polypeptides. Other interface residues are those at positions 6, 7, 11-14, 32-37, and 58-61 relative to the reference polypeptides. Thus, in these embodiments, at least 1, 2, 3, 4, 5, 6, 7, 8, 9, 10, 11, 12, 13, 14, 15, or all 16 of residues 6, 7, 11-14, 32-37, and 58-61 are identical relative to the reference polypeptide. As noted above, the position of residues 8-10 in the polypeptides of the disclosure is relative to the reference polypeptide and does not necessarily mean that residues 8-10 of the polypeptides of the disclosure will be RGD. Those of skill in the art will understand that the polypeptides of the disclosure may be fused to other functional domains (such as the fusion proteins or polypeptides with insertions described below), including N-terminal domains, such that the RGD residues in the polypeptides will not be at positions 8-10, but will still have the RGD triad at residues 8-10 relative to the reference polypeptide selected from SEQ ID NO:1-14.

TABLE 2

| | | | | | Protein | Loop |
|---|---|---|---|---|---|---|
| Position | Option 1 | Option 2 | Option 3 | Interface | Core | Insertion |
| 1 | All amino acid | F, M, W, I, V, | S, T | | | X |
| | | L, A, N, C, Q, | | | | |
| | | G, S, T, Y, R, | | | | |
| | | D, E, H, K | | | | |
| 2 | Charged or V | V, E, D | E, D | | | X |
| 3 | M, A, I, L, V | M, A, I, L, V | I, L, V | | | |
| 4 | G, E, I, V | E, I, V | I, V | | | |
| 5 | L, M, A, I, V | L, M, A, I, V | I, V | | X | |
| 6 | S, Q, N, H | S, Q, N, H | H | X | | |
| 7 | M, I, V, L, A, | Y, F, G | G | X | | |
| | Y, F, G | | | | | |
| 8 | R | R | R | X | | |
| 9 | G | G | G | X | | |
| 10 | D | D | D | X | | |
| 11 | M, F, I | M, F, I | F, I | X | | |
| 12 | G, P | G, P | P | X | | |
| 13 | Hydrophilic, R, | G, T, Y, H, K, | S, R | X | | |
| | K, H | S, R | | | | |
| 14 | A, T, S | T, S | S | X | | |
| 15 | N, Q, H, P, E, S | P, E, S | E, S | | X | |
| 16 | I, V, R, L, M | I, V, R, L, M | L, M | | X | |
| 17 | Hydrophilic, | V, G, T, Y, R, | E, A | | | |
| | charged, V, A | H, D, E, A | | | | |
| 18 | Hydrophilic, | H, D, Q, T, N, | R, K, L | | X | |
| | charged, I, V | I, V, R, K, L | | | | |
| 19 | Hydrophobic, E | E, W, M, I, L, F | L, F | | X | |
| 20 | Hydrophilic | E, K, R, Y, M, | L, F | | | |
| | other than G, | Q, L, F | | | | |
| | charged, L, F, | | | | | |
| | M | | | | | |
| 21 | H, T, V, G, E, K | H, T, V, G, E, K | E, K | | | |
| 22 | H, S, T, W, M, | H, S, T, W, M, | E, R, V | | | |
| | E, R, V | E, R, V | | | | |
| 23 | H, Y, S, M, I, | H, Y, S, M, I, V | F, L, A | | | |
| | V, F, L, A | | | | | |
| 24 | Hydrophilic, | D, R, H, E, K | E, K | | | |
| | charged | | | | | |
| 25 | Hydrophilic, | V, N, K, C, E, R | E, R | | | |
| | charged, V | | | | | |
| 26 | Q, R, S, T, C, | Q, R, S, T, C, | L, K | | | X |
| | L, K | L, K | | | | |
| 27 | Hydrophilic | D, N, C, G | G | | | X |
| 28 | Hydrophilic, I, | H, N, T, Y, V, | I, L | | | X |
| | L, V | C, I, L | | | | |
| 29 | H, R, M, C, E, K | H, R, M, C, E, K | E, K | | | |
| 30 | Hydrophobic | L, I, M, V | V | | X | |
| 31 | Hydrophilic, | D, K, E, R | E, R | | | |
| | charged | | | | | |
| 32 | Hydrophobic, | I, L, V, R | V, R | X | | |
| | K, R | | | | | |
| 33 | E, Q, H, R, G, | E, Q, H, R, G, | D, K, N | X | | |
| | D, K, N | D, K, N | | | | |
| 34 | S, Y, F, Q, H | S, Y, F, Q, H | H | X | | |
| 35 | S, N, K, R | S, N, K, R | K, R | X | | |
| 36 | D, N, E, Q, V, T | D, N, E, Q, V, T | V, T | X | | |
| 37 | I, M, A, L, V | I, M, A, L, V | L, V | X | | |
| 38 | All amino acids | D, E, K, R, T, H | T, H | | | |
| 39 | Hydrophobic, | M, N, C, L, V, A | L, V, A | | X | |
| | N, C | | | | | |
| 40 | Hydrophilic, | L, Q, K, I | I, K | | | X |
| | charged, L, I | | | | | |
| 41 | Hydrophilic, | G | G | | | X |
| | charged | | | | | |
| 42 | M, A, I, L, V | M, A, I, L, V | I, L, V | | | X |
| 43 | Hydrophilic, | T, S | S | | | X |
| | charged | | | | | |
| 44 | Hydrophilic, | H, D, C, K, E | K, E | | | X |
| | charged | | | | | |
| 45 | Hydrophilic, | Q, K, E | E | | | |
| | charged | | | | | |
| 46 | Hydrophilic, | F, M, I, L, V, | E, Q | | | |
| | charged, F, M, | E, Q | | | | |
| | I, L, V | | | | | |
| 47 | L, V, M, A, I | L, V, M, A, I | A, I | | | |
| 48 | Hydrophilic, | N, Q, G, S, T, | E, R | | | |
| | charged | Y, D, K, E, R | | | | |
| 49 | Hydrophilic, | K, I, E, R | E, R | | | |
| | charged, I | | | | | |
| 50 | Hydrophobic, Y | M, F, Y, L | L | | | |
| 51 | Y, C, A, E | Y, C, A, E | A, E | | | |
| 52 | Hydrophilic, | E, Q, R, K | R, K | | | |
| | charged | | | | | |
| 53 | Any amino acid | E, V | E, V | | | |
| | other than P | | | | | |
| 54 | Hydrophobic, | I, W, K, C, L, A | L, A | | | |
| | K, C | | | | | |
| 55 | W, Y, F, R, K, E | W, Y, F, R, K, E | R, K, E | | | |
| 56 | Hydrophilic, | R, T, S, K | S, K | | | |
| | charged | | | | | |
| 57 | Hydrophilic, | H, R, L | R, L | | | |
| | charged, L | | | | | |
| 58 | L, I, V, A, P, G | L, I, V, A, P, G | G | X | | |
| 59 | F, I, V, L | F, I, V, L | I, V, L | X | | |
| 60 | Y, F, W | Y, F, W | W | X | | |
| 61 | I, L, S, C, V, T | S, C, V, T | V, T | X | | |
| 62 | Hydrophilic, | I, Y, R, K, E, L | E, L | | | |
| | charged, I, L | | | | | |
| 63 | M, I, V | M, I, V | I, V | | X | |
| 64 | Hydrophilic, | M, T, R, K | R, K | | | X |
| | charged, M | | | | | |
| 65 | Any amino acid | P, G, S, R, K, | K, E, V | | | X |
| | | E, V | | | | |

Key:
Charged: R, D, E, H, K
Hydrophilic: N, C, Q, G, S, T, Y
Hydrophobic: F, M, W, I, V, L, P, A
Small hydrophobic: I, L, V, M, A, P
Any amino acid: R, D, E, H, K, N, C, Q, G, S, T, Y, F, M, W, I, V, L, P, A

In another embodiment, at least 1, 2, 3, 4, 5, 6, 7, or all 8 core residues are identical in the polypeptide relative to those in the reference polypeptide. Table 2 shows the position of core residues in the reference polypeptides, which are present at positions 5, 15, 16, 18, 19, 30, 39, and 63. The core residues are involved in overall structure of the reference polypeptides. Thus, in these embodiments, at least 1, 2, 3, 4, 5, 6, 7, or all 8 of residues 5, 15, 16, 18, 19, 30, 39, and 63 are identical relative to the reference polypeptides.

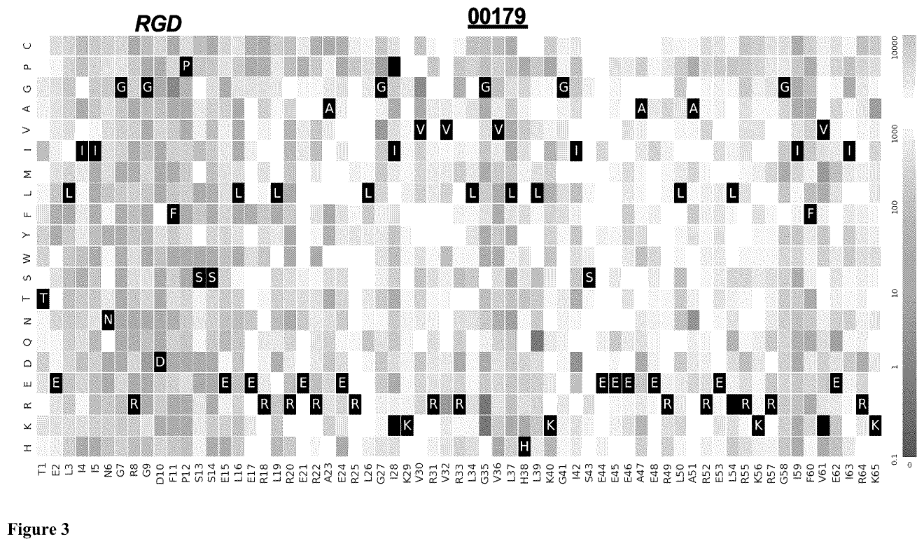

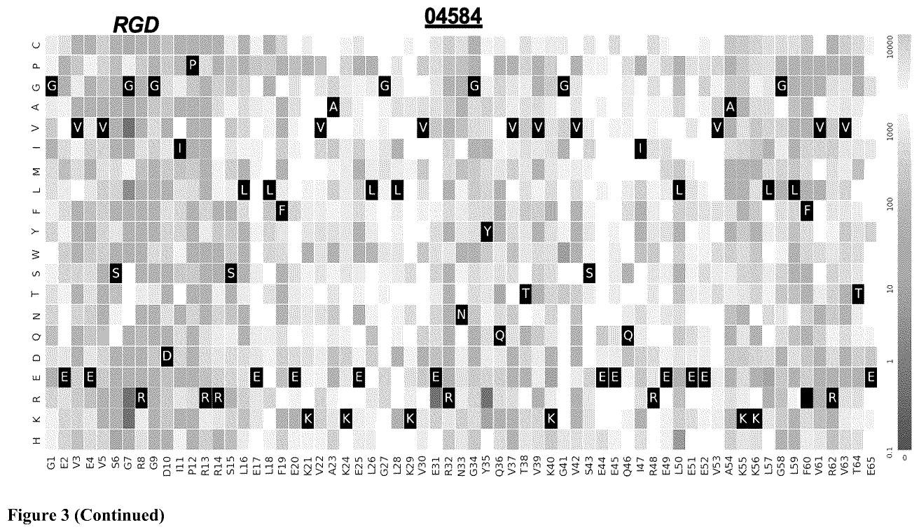

In a further embodiment, substitutions relative to the reference polypeptide are selected from those listed in Table 2, which lists Options 1, 2, and 3 for residues at different positions relative to the reference polypeptide, based on site saturation mutagenesis (SSM) studies described in the examples (FIG. 3 for exemplary SSMs). Underneath Table 2 is a key is provided to define classes of amino acid residues referred to in Table 2. In this embodiment, substitutions for a given residue may be any amino residue shown in a given row under any of Options 1, 2, or 3 listed at that position. In this embodiment, for example, position 17 relative to the reference polypeptide may be (a) hydrophilic or charged amino acids in Option 1 (N, C, Q, G, S, T, Y, R, D, E, H, or K); (b) V, G, T, Y, R, H, D, E, A in Option 2; or (c) E or A in Option 3. Where the class of residues in Option 1 includes two options separated by a comma, this means that Option 1 residues can be any residues in either of the listed groups.

It will be clear to those of skill in the art based on the teachings herein what residues are permissible at other positions relative to the reference polypeptide in Options 1, 2, or 3 in Table 2.

In other embodiments, relative to the reference polypeptide, 1, 2, 3, 4, 5, 6, 7, 8, 9, 10, 11, 12, or all 13 of the following are true:
Residue 3 is L or V;
Residue 5 is I or V;
Residue 6 is H;
Residue 13 is S or R;
Residue 14 is S;
Residue 31 is E or R;
Residue 32 is V or R;
Residue 33 is D, K, or N;
Residue 34 is H;
Residue 35 is K or R;
Residue 58 is G;
Residue 59 is I, V, or L; and/or
Residue 60 is W.

The site saturation mutagenesis and other studies described in the examples indicate that these residues help provide maximal integrin α5β1 binding affinity and/or specificity, and/or protein stability.

In further embodiments, relative to the reference polypeptide, 1, 2, 3, 4, 5, 6, 7, or all 8 of the following are true:
Residue 31 is E or R;
Residue 32 is V or R;
Residue 33 is D, K, or N;
Residue 34 is H;
Residue 35 is K or R;
Residue 58 is G;
Residue 59 is I, V, or L; and/or
Residue 60 is W.

In another embodiment, residue 58 relative to the reference polypeptide is G. In a further embodiment, residue 12 relative to the reference polypeptide is P.

In one embodiment, substitutions relative to the reference polypeptide are conservative amino acid substitutions. As used here, “conservative amino acid substitution” means that:
hydrophobic amino acids (Ala, Cys, Gly, Pro, Met, Sce, Sme, Val, Ile, Leu) can only be substituted with other hydrophobic amino acids;
hydrophobic amino acids with bulky side chains (Phe, Tyr, Trp) can only be substituted with other hydrophobic amino acids with bulky side chains;
amino acids with positively charged side chains (Arg, His, Lys) can only be substituted with other amino acids with positively charged side chains;
amino acids with negatively charged side chains (Asp, Glu) can only be substituted with other amino acids with negatively charged side chains; and
amino acids with polar uncharged side chains (Ser, Thr, Asn, Gln) can only be substituted with other amino acids with polar uncharged side chains.

In a further embodiment, the polypeptides may comprise an insertion in one or more loop regions of the polypeptide. The insertion may be any one or more amino acid, and may comprise a functional domain as described in the next paragraph, or one or more amino acids for additional spacing or for any other purpose. In one embodiment, an insertion in the loop regions is 1-3, 1-2, 1, 2, or 3 amino acids in length. Table 2 also shows the position of loop regions in the polypeptides of the disclosure (see column 2). In certain embodiments, amino acids or amino acid domains (such as a functional domain) may be inserted in the loop region (see column 2). As shown in Table 2, loop residues in the reference sequences are residues 1-2, 26-29, 40-44, and 64-65. The polypeptides of the disclosure may include any such insertion, and in these embodiments the polypeptide would still comprise the reference amino acid sequence, with an interruption at the site of insertion.

In another embodiment, the disclosure provides fusion proteins, comprising a polypeptide of any embodiment or combination of embodiments herein, fused to one or more functional domains. In these embodiments, any functional domain may be inserted, as an insertion at a loop region, and/or at the N-terminus and/or at the C-terminus of the polypeptide. In various non-limiting embodiments, the functional domain may comprise, for example, a targeting domain, a detectable domain, a scaffold domain such as a hetero/homo oligomer, a secretion signal, an Fc domain, or a further therapeutic peptide domain. In all embodiments, the fusion protein may optionally comprise an amino acid linker separating the polypeptide and the functional domain. Any amino acid linker may be used as suitable for an intended purpose, including but not limited to a GS-rich linker.

In one embodiment, the functional domain comprises domain scaffold domain. Any scaffold domain may be used as appropriate for an intended purpose. In one non-limiting embodiment, the scaffold domain comprises the amino acid sequence of SEQ ID NO:18, wherein the N-terminal methionine residue may be present or may be deleted.

(SEQ ID NO: 18)
(M)SGRQEKVLKSIEETVRKMGVTMETHRSGNEVKVVIKGLHIKQ
QRQLYRDVRETSKKQGVETEIEVEGDTVTIVVRE

In one such embodiment, the resulting fusion protein may comprise the amino acid sequence of SEQ ID NO:16, wherein all residues in parentheses are optional and may be present or may be deleted. In this embodiment, the polypeptide of SEQ ID NO:14 is fused to the scaffold of SEQ ID NO:18 via a GGSGG (SEQ ID NO:19) linker. It will be apparent to those of skill in the art based on the teachings herein, that the polypeptide of any embodiment disclosed herein may similarly be fused to the scaffold of SEQ ID NO:18.

1. 00179_v4.1_N-term-LHD101A
(SEQ ID NO: 16)
(M)SGRQEKVLKSIEETVRKMGVTMETHRSGNEVKVVIKGLHIKQ
QRQLYRDVRETSKKQGVETEIEVEGDTVTIVVREGGSGGSELIIH
GRGDFPRSELERLRERFERLGIKVRVDHKVLTLIGISEEEAERLA
RELRKRGIWVEIRK(GSGSHHWGSTHHHHHH)

In this embodiment, the fusion protein of SEQ ID NO:16 may be mixed with a second scaffold protein comprising the amino acid sequence of SEQ ID NO:17 wherein all residues in parentheses are optional and may be present or may be deleted, resulting in non-covalent interaction of the two scaffold domains, forming a hexamer, as described in the examples that follow.

2. C6-71-9-1_101B (hexameter-monomer-LHD101B)
(SEQ ID NO: 17)
(M)GRQEKVLKSIEETVRKMGVTMETHRSGNEVKVVIKGLHESQQ
EQLLEDVLRTAEKQGVRVRIRFKGDTVTIVVREGSPEEILERAKE
SLERAKEAFERGDEEEFRKAAEKALELAKRLVEQAKKEGDPELVF
EAARVALWVAWLAAWFGDKEVFKKAAESALEVAKRLVEVAKEEGD
PELVLKAAFVALLVAIMAVILGDKEVFKKAAESALEVAKRLVEIA
AREGDPELVEEAAKVAELVRELAKLMGDEEVYEKARETAREVRLF
LLFVRIWEGGGGS(LEHHHHHH)

In a further embodiment of any of the above embodiments, the polypeptide or fusion protein binds integrin α5β1, such as human integrin α5β1, with nanomolar affinity as determined by biolayer interferometry (BLI) as disclosed in the examples that follow. In some embodiments, the polypeptide or fusion protein binds integrin α5β1, such as human integrin α5≈1 with an affinity between 0.3 nm to 100 nm as determined by BLI using a buffer comprising 20 mM Tris, 150 mM NaCl, 1 mM MgCl2, 1 mM CaCl2, at pH 7.4.

In another aspect the disclosure provides nucleic acids encoding the polypeptide or fusion protein of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide or fusion protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptide or fusion protein of the disclosure.

In all embodiments, the percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, such as the Needleman and Wunsch (J. Mol. Biol. (48):444-453 (1970)) algorithm which has been incorporated into the GAP program in the GCG software package (available at http://www.gcg.com), the NBLAST and XBLAST programs (version 2.0) of Altschul, et al. (1990) J. Mol. Biol. 215:403-10, both of which are incorporated herein by reference in their entireties.

In a further aspect, the disclosure provides expression vectors comprising the nucleic acid of any aspect of the disclosure operatively linked to a suitable control sequence, such as a promoter. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the polypeptide, fusion protein, nucleic acid, or expression vector (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In a further embodiment, the disclosure provides pharmaceutical compositions, comprising:
(a) the polypeptide, the nucleic acid, the expression vector, and/or the host cell of any preceding claim; and
(b) a pharmaceutically acceptable carrier.

In certain embodiments, the carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising an immunomodulatory fusion protein is prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising an immunomodulatory fusion protein is formulated as a lyophilizate using appropriate excipients such as sucrose.

The compositions may be used, for example, in the methods disclosed herein. The compositions may further comprise for example, (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

The polypeptide, fusion protein, nucleic acid, expression vector, and/or host cell may be the sole active agent in the composition, or the composition may further comprise one or more other agents suitable for an intended use. In various non-limiting embodiments, such other agents may include angiogenesis inhibitors (including but not limited to axitinib, bevacizumab, cabozantinib, everolimus, lenalidomide, lenvatinib mesylate, pazopanib, ramucirumab, regorafenib, sorafenib, sunitinib, thalidomide, vandetanib, and ziv-aflibercept), immune checkpoint inhibitors (including, but not limited to, pembrolizumab, nivolumab, and cemiplimab as anti-PD-1 antibodies, ipilimumab as an anti-CTLA-4 antibody, and atezolizumab, avelumab, and durvalumab as anti-PD-L1 antibodies), and other cancer growth inhibitors including but not limited to tyrosine kinase inhibitors (including but not limited to alectinib, brigatinib, ceritinib, crizotinib, entrectinib, lorlatinib, ALK, I, bosutinib, dasatinib, imatinib, nilotinib, ponatinib, afatinib, dacomitinib, erlotinib, gefitinib, lapatinib, neratinib, osimertinib, vandetanib, gilteritinib, midostaurin, erdafitinib, ruxolitinib, larotrectinib, axitinib, carbozantinib, lenvatinib, pazopanib, regorafenib, sorafenib, sunitinib, dabrafenib, encorafenib, vemurafenib, acalabrutinib, ibrutinib, binimetinib, cobimetinib, trametinib, abemaciclib, palbociclib, or ribociclib), proteasome inhibitors (including but not limited to ortezomib, carfizomib, ixazomib, delanzomib, oprozomib, and marizomib), mTOR inhibitors (including but not limited to everolimus, sirolimus, temsirolimus, everolimus, sirolimus, sirolimus protein-bound, and everolimus), PI3K inhibitors (including but not limited to copanlisib, alpelisib, idelalisib, duvelisib and umbralisib), histone deacetylase inhibitors (including but not limited to vorinostat, romidepsin, panobinostat, and belinostat), and Hedgehog pathway blockers (including but not limited to vismodegib, sonidegib, and glasdegib).

In another aspect, the disclosure provides methods for treating cancer or a vascular disease, comprising administering to a subject in need thereof an amount effective of the polypeptide, fusion protein, nucleic acid, expression vector, host cell, and/or pharmaceutical composition of any embodiment disclosed herein to treat the cancer or the vascular disease. The overexpression of integrin α5β1 is closely linked to the severe progression of multiple human cancers due to its role in regulating cell migration and proliferation. See, for example, Hou et. al., Onco Targets Ther. 2020; 13: 13329-13344, 2019, which is incorporated by reference herein in its entirety, for the role of integrin α5β1. The polypeptides of the disclosure are shown to slow down cancer cell migration and attachment, and thus be useful, for example, in treating cancers by stopping tumor cells from spreading and preventing angiogenesis. Integrin α5β1 overexpression is also observed in several vascular diseases and autoimmune diseases, and thus the polypeptides of the disclosure can also be used, for example for treating vascular diseases such as diabetic retinopathy, choroidal neovascularization, stroke, pulmonary arterial hypertension, and rheumatoid arthritis.

In one embodiment, the methods are for treating cancer, and comprise administering to a subject having cancer an amount of the polypeptide, fusion protein, nucleic acid, expression vector, host cell, and/or pharmaceutical composition of any preceding claim effective to treat the cancer.

In another embodiment, the methods are for inhibiting tumor metastasis, and comprise administering to a subject having cancer an amount of the polypeptide, fusion protein, nucleic acid, expression vector, host cell, and/or pharmaceutical composition of any preceding claim effective to inhibit tumor metastasis. In one embodiment, the methods comprising inhibiting tumor cell attachment to the extracellular matrix to inhibit tumor metastasis.

In a further embodiment, the methods are for inhibiting cell migration and/or attachment, and comprise administering to a subject in need thereof an amount effective of the polypeptide, fusion protein, nucleic acid, expression vector, host cell, and/or pharmaceutical composition of any embodiment disclosed herein to inhibit cell migration and/or attachment. In some embodiments, the cell is a tumor cell and/or an endothelial cell. The examples show that the polypeptides and/or fusion proteins of the disclosure can be used to inhibit human umbilical vein endothelial cell (HUVEC) migration and tube formation. The polypeptides and/or fusion proteins of the disclosure have also been shown to inhibit, for example, HUVEC attachment to fibronectin-containing extracellular matrix (data not shown). In particular, the polypeptides and/or fusion proteins of the disclosure comprising binder 00179_v4.1 (SEQ ID NO:12) or a hexamer comprising 00179_v4.1_N-term-LHD101A (SEQ ID NO:16) and C6-71-9-1_101B (hexameter-monomer-LHD101B) (SEQ ID NO:17) can be used to inhibit human umbilical vein endothelial cell (HUVEC) migration and tube formation. Thus, in one embodiment, the methods inhibit cell migration or attachment of HUVECs. In another embodiment, the methods inhibit cell attachment, such as HUVEC attachment, to fibronectin in extracellular matrix. In still other embodiments, the methods inhibit cell migration or attachment of HUVECs, and the polypeptide, fusion protein, nucleic acid, expression vector, host cell, and/or pharmaceutical composition comprises 00179_v4.1 (SEQ ID NO:12) or a hexamer comprising 00179_v4.1_N-term-LHD101A (SEQ ID NO:16) and C6-71-9-1_101B (hexameter-monomer-LHD101B) (SEQ ID NO:17). In these embodiments, the methods can be used, for example, to treat cancer and/or vascular disease accompanied by angiogenesis.

In another embodiment, the methods are for treating cancer, and comprise administering to a subject undergoing radiation therapy to treat cancer an amount effective of the polypeptide, fusion protein, nucleic acid, expression vector, host cell, and/or pharmaceutical composition of any embodiment disclosed herein to enhance effectiveness of the radiation therapy. Integrin α5β1 inhibition can help to improve effectiveness of radiation therapy (see, for example, Yao et al., Translational Oncology, Volume 4, Issue 5, October 2011, Pages 282-292, hereby incorporated by reference in its entirety), and thus the methods of this embodiment can enhance effectiveness of radiation therapy. Any enhancement in radiation therapy efficacy would be of significant benefit to patient treatment, and may include reduced side effects, improved therapeutic outcome, ability to limit radiation dosage, etc. The methods may be used with any radiation therapy protocol, including but not limited to external beam radiation therapy and targeted radiotherapy, and at any radiation dose.

In any of these methods involving treating cancer or limiting tumor metastasis, tumor cell migration, or tumor cell attachment, the subject may have any cancer that attending medical personnel believe can be treated using the methods of the disclosure. In various non-limiting embodiments, the cancer is selected from the group consisting of glioblastoma, glioma, colon cancer, colorectal cancer, lung cancer, breast cancer, prostate cancer, ovarian cancer, cervical cancer, Ewing sarcoma, acute lymphoblastic leukemia, melanoma, basal cell carcinoma, multiple myeloma, osteosarcoma, squamous carcinoma, head and neck squamous cell carcinoma, mesothelioma, pancreatic ductal adenocarcinoma, gastric cancer, cholangiocarcinoma, epidermoid carcinoma, chondrosarcoma, neuroblastoma, rectal cancer, and transitional carcinoma.

In another embodiment, the methods are for treating a vascular disease, comprising administering to a subject having a vascular disease an amount effective of the polypeptide, fusion protein, nucleic acid, expression vector, host cell, and/or pharmaceutical composition of any embodiment disclosed herein to treat the vascular disease. The methods may be used to treat any suitable vascular disease, such as those in which inhibiting angiogenesis would be therapeutically beneficial, including but not limited to vascular diabetic retinopathy, choroidal neovascularization, stroke, and pulmonary arterial hypertension.

In a further embodiment, the methods are for treating rheumatoid arthritis, comprising administering to a subject having rheumatoid arthritis an amount effective of the polypeptide, fusion protein, nucleic acid, expression vector, host cell, and/or pharmaceutical composition of any embodiment disclosed herein to treat the rheumatoid arthritis.

In a further embodiment, the methods are for treating angiogenesis, comprising administering to a subject having a disorder associated with pathogenic angiogenesis an amount effective to limit the pathogenic angiogenesis of the polypeptide, fusion protein, nucleic acid, expression vector, host cell, and/or pharmaceutical composition of any embodiment disclosed herein. Uncontrolled (“pathogenic”) angiogenesis can lead to various disorders. Tumor angiogenesis is positively correlated with tumor malignancy. Pathogenic angiogenesis associated with diabetic retinopathy can result in insufficient retinal perfusion and hemorrhage. In atherosclerosis, oxidative stress and anoxia may lead to pathogenic angiogenesis from preexisting blood vessels. Angiogenesis is also involved in the onset of arthritis, such as rheumatoid arthritis. Choroidal neovascularization (pathogenic angiogenesis) is a feature of wet age-related macular degeneration. (See, for example, La Mondola et al., Int. J. Mol. Sci. 2022 September; 23(18): 10962; Nakagawa et al., Diabetes. 2009 July; 58(7): 1471-1478; and Helotera et al., Cells. 2022 November; 11(21): 3453, each of which is incorporated herein by reference in its entirety.) Thus, in some embodiments the disorder associated with pathogenic angiogenesis may be selected from the group consisting of macular degeneration (such as wet age-related macular degeneration), diabetic retinopathy, rheumatoid arthritis, atherosclerosis, and cardiovascular diseases.

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “inhibit” or “inhibiting” means reducing the amount, rate, and/or rate of increase of what is being inhibited (i.e., metastasis or cell migration and/or attachment).

When the method comprises treating cancer, “treat” or “treating” means accomplishing one or more of the following: (a) reducing the size or volume of tumors and/or metastases in the subject; (b) limiting any increase in the size or volume of tumors and/or metastases in the subject; (c) increasing survival; (d) reducing the severity of symptoms associated with cancer; (e) limiting or preventing development of symptoms associated with cancer; and (f) inhibiting worsening of symptoms associated with cancer. The methods can be used to treat any suitable cancer, including but not limited to glioblastoma, glioma, colon cancer, colorectal cancer, lung cancer, breast cancer, prostate cancer, ovarian cancer, cervical cancer, Ewing sarcoma, acute lymphoblastic leukemia, melanoma, basal cell carcinoma, multiple myeloma, osteosarcoma, squamous carcinoma, head and neck squamous cell carcinoma, mesothelioma, pancreatic ductal adenocarcinoma, gastric cancer, cholangiocarcinoma, epidermoid carcinoma, chondrosarcoma, neuroblastoma, rectal cancer, and transitional carcinoma. See, for example, Hou et al., Onco Targets Ther. 2020; 13: 13329-13344, 2019, which is incorporated herein by reference in its entirety.

The subject may be any subject that has a relevant disorder. In one embodiment, the subject is a mammal, including but not limited to humans, dogs, cats, horses, cattle, etc.

As used herein, an “effective” amount refers to an amount of the polypeptide, fusion protein, nucleic acid, expression vector, and/or host cell that is effective for treating the disorder. The polypeptides, fusion proteins nucleic acids, expression vectors, and/or host cells are typically formulated as a pharmaceutical composition, such as those disclosed above, and can be administered via any suitable route, including but not limited to orally, by inhalation spray, ocularly, intravenously, subcutaneously, intraperitoneally, and intravesicularly in dosage unit formulations containing conventional pharmaceutically acceptable carriers, adjuvants, and vehicles.

Any suitable dosage range may be used as determined by attending medical personnel. Dosage regimens can be adjusted to provide the optimum desired response. A suitable dosage range for the polypeptides or fusion proteins may, for instance, be 0.1 ug/kg-100 mg/kg body weight; alternatively, it may be 0.5 ug/kg to 50 mg/kg; 1 ug/kg to 25 mg/kg, or 5 ug/kg to 10 mg/kg body weight. In some embodiments, the recommended dose could be lower than 0.1 mcg/kg, especially if administered locally (such as by intra-tumoral injection). In other embodiments, the recommended dose could be based on weight/m² (i.e. body surface area), and/or it could be administered at a fixed dose (e.g.,.05-100 mg). The polypeptides, fusion proteins, nucleic acids, expression vectors, and/or host cells can be delivered in a single bolus, or may be administered more than once (e.g., 2, 3, 4, 5, or more times) as determined by an attending physician.

The polypeptides, fusion proteins, nucleic acids, expression vectors, and/or host cells made be administered as the sole therapeutic agent, or may be administered together with (i.e.: combined or separately) one or more other therapeutic agents, including but not limited to tumor resection, chemotherapy, radiation therapy, and immunotherapy (such as checkpoint inhibitors). In various non-limiting embodiments, other agents that may be administered in the methods of the disclosure include angiogenesis inhibitors (including but not limited to axitinib, bevacizumab, cabozantinib, everolimus, lenalidomide, lenvatinib mesylate, pazopanib, ramucirumab, regorafenib, sorafenib, sunitinib, thalidomide, vandetanib, and ziv-aflibercept), immune checkpoint inhibitors (including, but not limited to, pembrolizumab, nivolumab, and cemiplimab as anti-PD-1 antibodies, ipilimumab as an anti-CTLA-4 antibody, and atezolizumab, avelumab, and durvalumab as anti-PD-L1 antibodies), and other cancer growth inhibitors including but not limited to tyrosine kinase inhibitors (including but not limited to alectinib, brigatinib, ceritinib, crizotinib, entrectinib, lorlatinib, ALK, I, bosutinib, dasatinib, imatinib, nilotinib, ponatinib, afatinib, dacomitinib, erlotinib, gefitinib, lapatinib, neratinib, osimertinib, vandetanib, gilteritinib, midostaurin, erdafitinib, ruxolitinib, larotrectinib, axitinib, carbozantinib, lenvatinib, pazopanib, regorafenib, sorafenib, sunitinib, dabrafenib, encorafenib, vemurafenib, acalabrutinib, ibrutinib, binimetinib, cobimetinib, trametinib, abemaciclib, palbociclib, or ribociclib), proteasome inhibitors (including but not limited to ortezomib, carfizomib, ixazomib, delanzomib, oprozomib, and marizomib), mTOR inhibitors (including but not limited to everolimus, sirolimus, temsirolimus, everolimus, sirolimus, sirolimus protein-bound, and everolimus), PI3K inhibitors (including but not limited to copanlisib, alpelisib, idelalisib, duvelisib and umbralisib), histone deacetylase inhibitors (including but not limited to vorinostat, romidepsin, panobinostat, and belinostat), and Hedgehog pathway blockers (including but not limited to vismodegib, sonidegib, and glasdegib).

In another aspect, the disclosure provides medical devices or implants comprising a plurality of the polypeptide, fusion protein, or pharmaceutical composition of any preceding claim coated on or in the medical device or implant. The polypeptides of the disclosure have been shown to promote cell attachment and spreading of cells including human mesenchymal stem cells (hMSCs) and human fibroblasts, on a polypeptide-coated titanium implant and a hydrogel with polypeptide conjugated to the hydrogel (data not shown). Thus, the coated devices and/or implants may be used, for example, as biomaterials for regenerative medicine such as stem cell therapy.

### EXAMPLES

Efforts are described in the development of de novo α5β1-specific protein binders. Blueprint builder was used to generate a ferredoxin scaffolds library and graft the fibronectin RGD loop. Using high-throughput yeast display, high-affinity designed protein binders were identified targeting integrin α5β1 specifically. Using the designed mini-binders, binders are described that specifically inhibit cell attachment and migration, making them useful for inhibiting integrin α5β1 in relevant diseases.

### Results

### Using Blueprint Builder to Graft Fibronectin RGD Loop

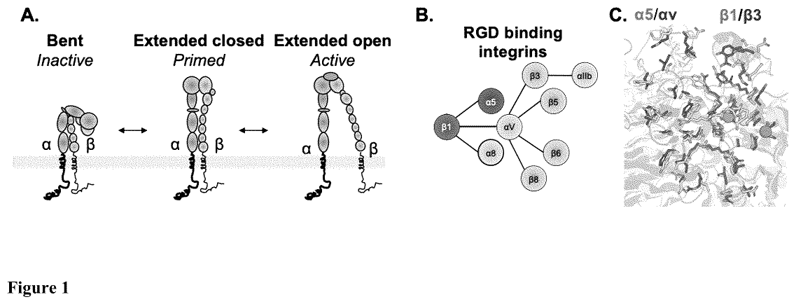

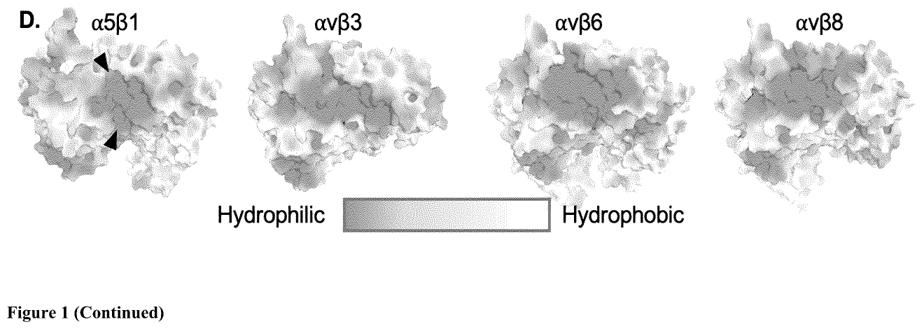

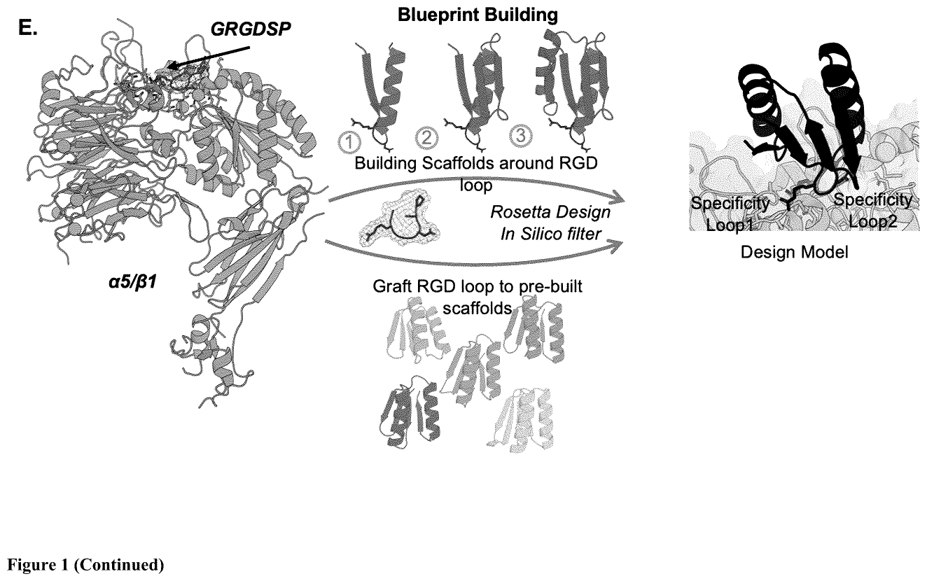

Integrin α5β1 undergoes conformational changes from bent to extended open when activated by its native ligand fibronectin. Fibronectin binds to Integrin α5β1 via a RGD motif (FIG. 1A). However, fibronectin can also bind to other RGD binding integrin members. (FIG. 1B, C). Designing integrin α5β1 binders has been challenging because of the high conservation of the RGD-binding integrin members. However, the area near the RGD binding site has mild differences including hydrophilic and hydrophobic residues distribution (FIG. 1D) which provide opportunities for specific binder development. We used a combination of computational design and experimental high-throughput screen for the development of the integrin α5β1 specific binders. (FIG. 1E, F). The design pipeline and binder model are shown in FIG. 1E.

Fibronectin interacts with integrin α5β1 through its RGD loop and a synergy site on β1. The RGD binding site is at the interface of the alpha and beta subunits. Therefore, proteins were designed to scaffold the RGD loop using the blueprint builder. Instead of assembling the whole ferredoxin fold in one step, a new method is proposed based on a piecewise blueprint builder. Using this method, small topologies (i.e.: alpha-alpha, alpha-beta-alpha, or beta-beta) pieces are grown from a starting structure one by one, restricting the position of each amino acid for the next growth step. The reason behind the use of this method is that producing small proteins is more efficient when using the blueprint builder, hence obtaining larger outputs when compared to producing complex structures in one step. In addition, the piecewise blueprint builder allows for checking the output obtained at each growing step and modifying the blueprint accordingly. Designs started from the crystal structure of human integrin α5β1 in complex with an RGD peptide (PDB 4WK2). An alpha helix was grown from the C-terminus and a beta strand from the N-terminus of RGD sampling different lengths using the blueprint builder, and the amino acids of the designs were then optimized at the binding interface by superimposing integrin α5β1 and selected those designs that presented the lowest energy interactions. In a second step, a beta-hairpin was grown, parallel to the first beta-strand, at the C-terminus of the alpha helix sampling different structures and loop lengths following the rules for designing ideal protein structures. After optimizing the amino acids at the binding sites and selecting the best designs, an alpha helix was grown followed by a beta strand, each one parallel to the first alpha helix and the first beta-strand, respectively. Interface optimization and selection of the best designs were performed before a final step in which the whole backbone except RGD was redesigned to obtain sequences with the lowest energy state.

However, due to the flexibility of the RGD loop, it was difficult to constrain its conformation in the built scaffolds. Therefore, a 20,000-member ferredoxin scaffold library was constructed, to match the C- and N-terminus of the RGD loop. The 5-present loops can serve as anchoring binding motifs and allow further engineering for affinity maturation. Using both methods, constructs were designed and filtered to ˜15,000 proteins and screened for binding using yeast display. By combining the beneficial mutations, combination libraries were constructed and screened to as low as 200 pico-molar.

### Integrin α5β1-Mb Binds to α5β Specifically and Tightly.

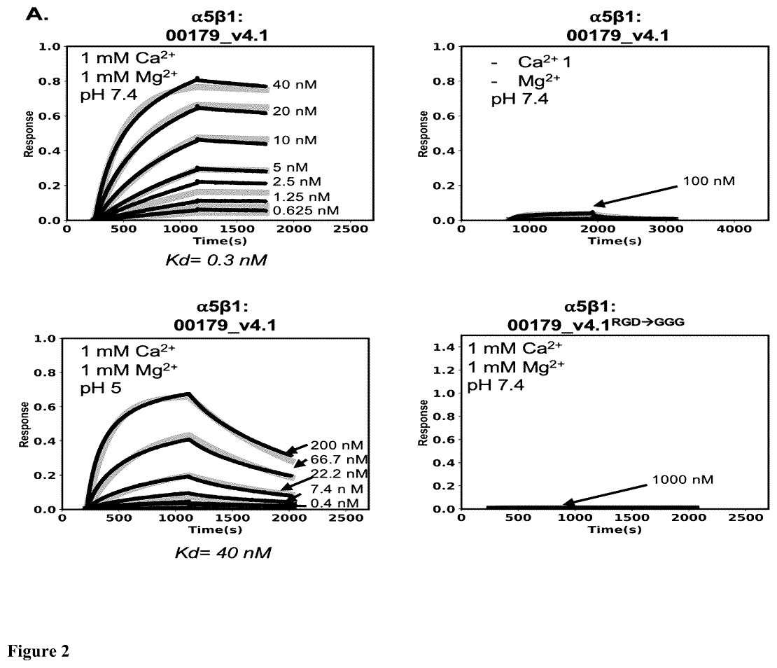

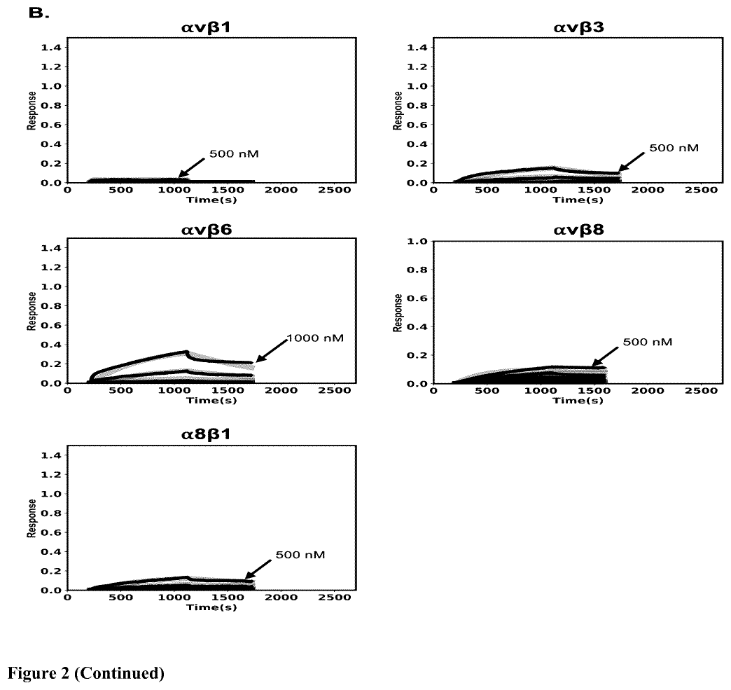

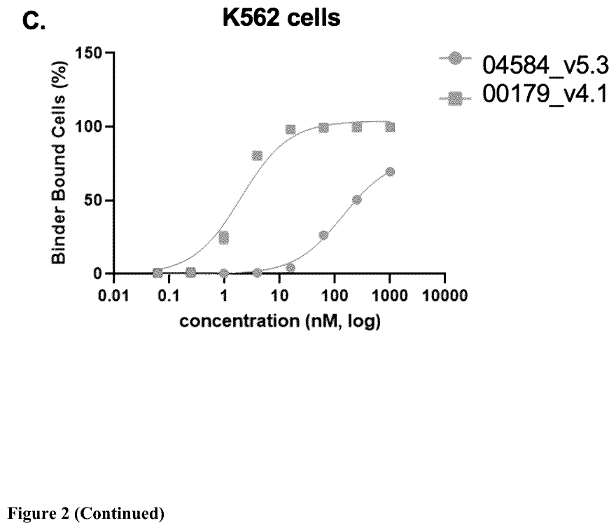

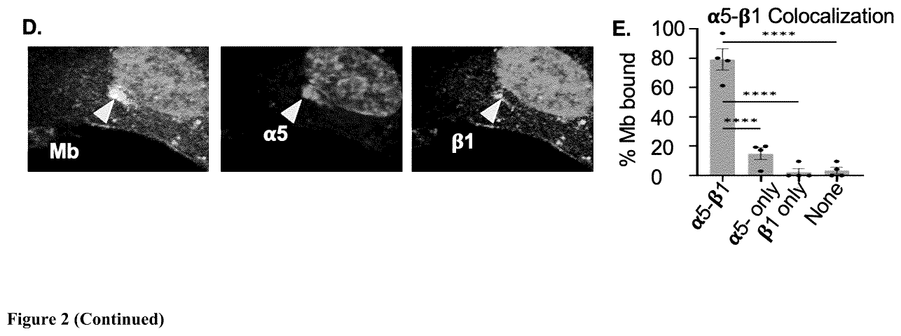

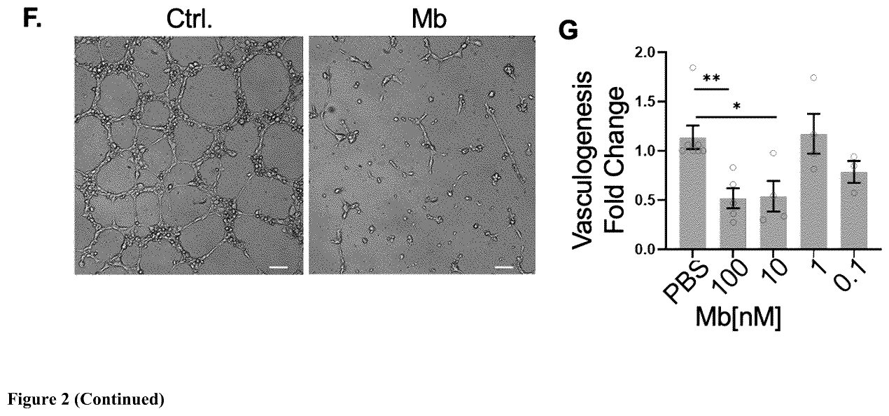

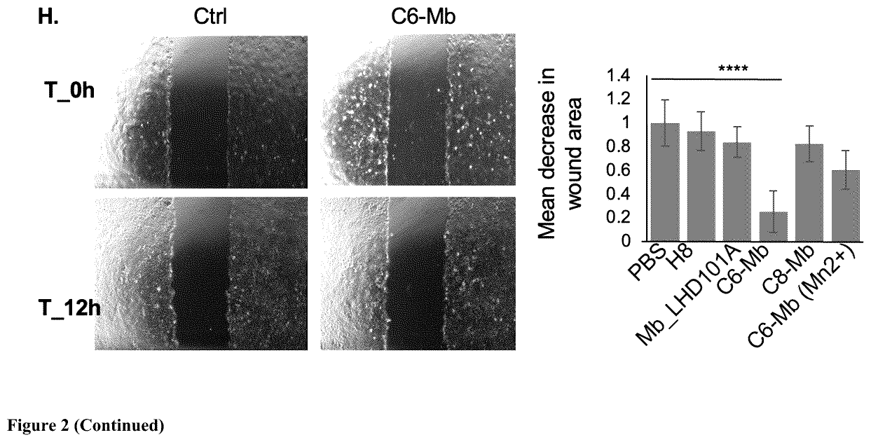

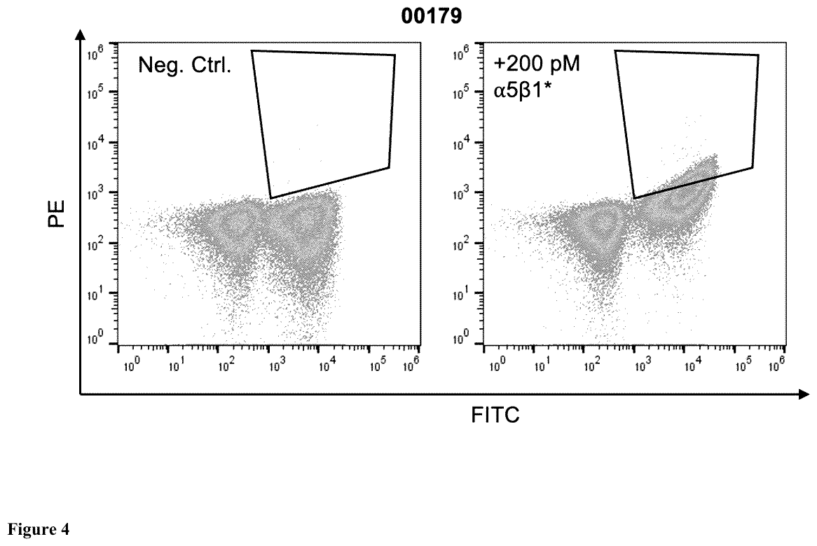

15 constructs of identified initial hits were expressed and purified—their binding affinities range from nanomolar to micromolar (data not shown). Saturated site mutation libraries were constructed and screened for binding to integrin α5β1 at different concentrations as well as excessive amount of integrin avb3 or a8b1 to select α5β1 preferred minibinder variants. The binding affinity of each variant was calculated as described in Cao et al, 2022. By combining the beneficial mutations, combination libraries were constructed and screened for binding to α5β1 at an affinity as low as 200 pico-molar (FIG. 4). Individual binder variants were then expressed and purified and assayed for their binding affinity to integrin α5β1 using biolayer interferometry (BLI) in integrin resting buffer (20 mM Tris pH 7.4, 150 mM NaCl, 1 mM MgCl₂, 1 mM CaCl₂, 0.02% tween-20). The highest binder 00179_v4.1 (SEQ ID NO:12) binds to integrin α5β1 with a KD=0.3±0.1 nM (FIG. 2A). Because RGD binding depends on metal ions, the binders were tested for binding to integrin without metal ions. Indeed, when no metal ion was present in the assay buffer, little binding was observed at 100 nM integrin α5β1. Similarly, mutating the RGD motif to GGG also abolished the binding ability. Surprisingly, binder 00179_v4.1 still can bind to α5β1 at pH 5 supplemented with 1 mM MgCl₂, 1 mM CaCl₂. This is surprising, and provides a tool to track integrin α5β1 in endosome and lysosome.

The highest affinity binder (00179_v4.1) also showed high specificity. No detectable binding of 00179_v4.1 was observed in 500 nM of integrin a8b1, avb1, avb6, avb8 and avb3 (FIG. 2B). Because of the large size of integrin and the distribution of integrin α5β1 on the cell surface, it was of interest to measure the affinity of the binders on the cell surface. Using wild type A562, the binding affinity of binders to α5β1 was measured, which is the only RGD binding integrin expressed in WT A562. Nanomolar binding ability was observed in the best binder (00179_v4.1) (FIG. 2C).

To investigate if the minibinder localizes with active α5β1, a spy-tagged minibinder was made and conjugated to a GFP-tagged sheet to aid visualization of minibinder localization. As expected, the GFP minibinder sheet only localizes with α5β1 but not α5 or β1 alone. Surprisingly, only the active β1 is recruited to the minibinder, suggesting that the minibinder binding stabilizes the active β1 conformation (FIGS. 2D and 2E).

### Soluble α5β1-Minibinder (Mb) Inhibits HUVEC Cell Tube Formation and Cell Migration.

Integrin α5β1 is highly upregulated in newly formed blood vessels and α5β1 integrins co-localize with Tie2 to promote angiogenesis. Genetic ablation of α5 integrin in mice causes severe vascular defects and increased permeability. Integrin α5β1 is also highly expressed in many cancers, including brain, colon, breast, and cervical cancers, which promotes tumor angiogenesis, cancer cell proliferation and metastasis. The capacity of integrin α5β1 minibinders to inhibit angiogenesis was evaluated using HUVECs in tube formation assay. 00179_v4.1 (SEQ ID NO:12) was seeded with HUVECs at 0.1 nM to 1000 nM or with phosphate buffered saline (PBS) as a positive control for 12 hours before images were taken for analysis (FIG. 2F). Quantification shows tube formation was significantly attenuated at 10 nM and 100 nM of α5β1-mb compared to PBS control (FIG. 2G). The data demonstrate α5β1-mb is a potent inhibitor of angiogenesis and can be used for inhibiting tumor angiogenesis and metastasis.

The inhibitory effect of 20 nM soluble 00179_v4.1 on cell migration was assessed using the scratch-wound assay. HUVECs were seeded with either 00179_v4.1 or PBS (used as a positive control) for 12 hours before capturing images for analysis. Quantitative results indicate a significant reduction in cell migration at the tested concentration of 00179_v4.1 compared to the PBS control. Furthermore, when 00179_v4.1 point mutant (SEQ ID NO:14) was conjugated with a hexamer, 20 nM C6-mb demonstrated an even more pronounced inhibition of cell migration (FIG. 2H).

The hexamer was made as follows (residues in parentheses are optional)

1. 00179_v4.1_N-term-LHD101A
(SEQ ID NO: 16)
(M)SGRQEKVLKSIEETVRKMGVTMETHRSGNEVKVVIKGLHIKQ
QRQLYRDVRETSKKQGVETEIEVEGDTVTIVVREGGSGGSELIIH
GRGDFPRSELERLRERFERLGIKVRVDHKVLTLIGISEEEAERLA
RELRKRGIWVEIRK(GSGSHHWGSTHHHHHH)
2. C6-71-9-1_101B (hexameter-monomer-LHD101B)
(SEQ ID NO: 17)
(M)GRQEKVLKSIEETVRKMGVTMETHRSGNEVKVVIKGLHESQQ
EQLLEDVLRTAEKQGVRVRIRFKGDTVTIVVREGSPEEILERAKE
SLERAKEAFERGDEEEFRKAAEKALELAKRLVEQAKKEGDPELVF
EAARVALWVAWLAAWFGDKEVFKKAAESALEVAKRLVEVAKEEGD
PELVLKAAFVALLVAIMAVILGDKEVFKKAAESALEVAKRLVEIA
AREGDPELVEEAAKVAELVRELAKLMGDEEVYEKARETAREVRLF
LLFVRIWEGGGGS(LEHHHHHH)

Component 1 and 2 are mixed at 6:1 ratio to form the hexamer. Residues in parentheses are cloning artifacts and are not required for activity or hexamer formation.

### Methods and Materials

### Protein Expression and Purification

Synthetic genes encoding designed proteins were purchased from Genscript or Integrated DNA Technologies (IDT) in the pET29b expression vector or as eBlocks™ (IDT) and cloned into customized expression vectors (Wicky et al. 2022) using golden gate cloning. A His6×tag was included either at the N-terminus or at the C-terminus as part of the expression vector. In some cases a TEV protease recognition site was introduced at the N-terminus after the histidine tag.

Proteins were expressed using autoinducing TBII media (Mpbio) supplemented with 50×5052 (25% [wt/vol] glycerol, 2.5% [wt/vol] D-(+)-glucose, 10% [wt/vol] α-lactose; final media concentration is 1×), 20 mM MgSO4, and trace metal mix in BL21 LEMO E. coli cells. Proteins were expressed under antibiotic selection at 37 degrees Celsius overnight or at 18-25 degrees Celsius overnight after initial growth for 6-8 h at 37 degrees Celsius. Cells were harvested by centrifugation at 4000×g and resuspended in lysis buffer (100 mM Tris pH 8.0, 200 mM NaCl, 50 mM Imidazole pH 8.0) containing protease inhibitors (Thermo Scientific) and Bovine pancreas DNaseI (Sigma-Aldrich) before lysis by sonication. One millimolar of the reducing agent TCEP was included in the lysis buffer for designs with free cysteines. Proteins were purified by Immobilized Metal Affinity Chromatography. Cleared lysates were incubated with 2-4 ml nickel NTA beads (Qiagen) for 20-40 minutes before washing beads with 5-10 column volumes of lysis buffer, 5-10 column volumes of high salt buffer (10 mM Tris pH 8.0, 1 M NaCl) and 5-10 column volumes of lysis buffer. Proteins were eluted with 10 ml of elution buffer (20 mM Tris pH 8.0, 100 mM NaCl, 500 mM Imidazole pH 8.0). His6×tags were cleaved by dialyzing IMAC elutions against 20 mM Tris pH 8.0, 100 mM NaCl, and 1 mM TCEP overnight in the presence of His6×tagged TEV protease followed by a second IMAC column to remove His6×-TEV and uncleaved protein.

All protein preparations were as a final step polished using size exclusion chromatography (SEC) on either Superdex™ 200 Increase 10/300GL or Superdex™ 75 Increase 10/300GL columns (Cytiva) using 20 mM Tris pH 8.0, 100 mM NaCl. The reducing agent TCEP was included (1 mM final concentration) for designs with free cysteines. For designs where a substantial void volume peak was present in addition to the monomer peak, the monomer peak was pooled and reinjected. Only designs where upon reinjection the void peak was mostly absent were further pursued. SDS-PAGE and LC/MS were used to verify peak fractions. Proteins were concentrated to concentrations between 0.5-10 mg/ml and stored at room temperature or flash frozen in liquid nitrogen for storage at −80. Thawing of flash-frozen aliquots was done at room temperature or 37 degrees Celsius. All purification steps from IMAC were performed at ambient room temperature.

### Biolayer Interferometry

Biolayer interferometry experiments were performed on an OctetRED96™ BLI system (ForteBio, Menlo Park, CA) at room temperature in integrin resting buffer (20 mM Tris pH 7.4, 150 mM NaCl, 1 mM MgCl₂, 1 mM CaCl₂, 0.02% tween-20) or active buffer (20 mM Tris pH 7.4, 150 mM NaCl, 1 mM MnCl₂, 0.2 mM CaCl₂, 0.02% tween-20) or inactive buffer (20 mM Tris pH 7.4, 150 mM NaCl, 0.02% tween-20) or low-pH buffer (20 mM Tris pH 5, 150 mM NaCl, 1 mM MgCl₂, 1 mM CaCl₂, 0.02% tween-20) buffer supplemented with 0.2 mg/ml bovine serum albumin (SigmaAldrich). Prior to measurements, streptavidin-coated biosensors were first equilibrated for at least 10 min in assay buffer. Protein binders with an N-terminal biotin were immobilized onto the biosensors by dipping them into a solution with 100 nM protein until the response reached between 10% and 50% of the maximum value followed by dipping sensors into fresh buffer to establish a baseline for 120 s. Titration experiments were performed at 25 degrees Celsius while rotating at 1000 rpm. Association of integrins was allowed by dipping biosensors in solutions containing designed protein diluted in buffer until equilibrium was approached followed by dissociation by dipping the biosensors into fresh buffer solution to monitor the dissociation kinetics. In the minibinder binding cross specificity assays each biotinylated minibinder was loaded onto streptavidin biosensors in equal amounts followed by 2 min of baseline equilibration. The association and dissociation with all the different binders were allowed for 900-1500 s for each step. Global kinetic or steady-state fits were performed on buffer-subtracted data using the manufacturer's software (Data Analysis 12.1) assuming a 1:1 binding model.

### Circular Dichroism Spectroscopy

CD spectra were recorded in a 1 mm path length cuvette at a protein concentration between 0.3-0.5 mg/mL on a J-1500 instrument (Jasco). For temperature melts, data were recorded at 222 nm between 4 and 94° C. every 2° C., and wavelength scans between 190 and 260 nm at 10° C. intervals starting from 4° C. Experiments were performed in 20 mM Tris pH 8.0, 20 mM NaCl. The high-tension (HT) voltage was monitored according to the manufacturer's recommendation to ensure optimal signal-to-noise ratio for the wavelengths of interest.

### Tube Formation Assay

Tube formation assay was perform using a previously described protocol. Briefly, passage 4 HUVECs were thawed onto a 10 cm 0.1% gelatin pre-coated plate and cultured until 80-90% confluent. Before cell seeding, 150 uL of 100% Matrigel™ is added to a pre-chilled 24-well plate to allow even spreading of the Matrigel™. The Matrigel™ plate was allowed to solidify at room temperature for 25 minutes. HUVECs were seeded at 150,000 cells per 350 uL in each well with PBS or α5β1-mb at 0.1 to 1000 uM, considering 500 uL as the total volume in each well. Cells were then imaged at 12-hour timepoint. Twenty images were taken in each well at random locations and images were analyzed using the Angiogenesis analyzer plugin in ImageJ™. An average of the number of nodes, meshes, and segments of the 20 images, and these three parameters were also averaged to calculate the vascular stability for each well.

Various embodiments of the present technology are set forth herein below in paragraphs 1 to 50:

Para. 1. A polypeptide comprising or consisting of an amino acid sequence at least 50%, 55%, 60%, 70%, 75%, 80%, 85%, 90%, 91%, 92%, 93%, 94%, 95%, 96%, 97%, 98%, 99%, or 100% identical to the amino acid sequence selected from SEQ ID NO:1-14, not including any insertions, wherein residues 8-10 relative to the reference polypeptide are RGD, and wherein the polypeptide selectively binds to α(5)β(1) integrin.

Para. 2. The polypeptide of Para. 1, comprising or consisting of an amino acid sequence at least 50%, 55%, 60%, 70%, 75%, 80%, 85%, 90%, 91%, 92%, 93%, 94%, 95%, 96%, 97%, 98%, 99%, or 100% identical to the amino acid sequence selected from SEQ ID NO:11-14, not including any insertions, wherein the polypeptide selectively binds to α(5)β(1) integrin.

Para. 3. The polypeptide of Para. 1, comprising or consisting of an amino acid sequence at least 80%, 85%, 90%, 91%, 92%, 93%, 94%, 95%, 96%, 97%, 98%, 99%, or 100% identical to the amino acid sequence selected from SEQ ID NO:11-14, not including any insertions, wherein the polypeptide selectively binds to α(5)β(1) integrin.

Para. 4. The polypeptide of any one of Paras. 1-5, wherein at least 1, 2, 3, 4, 5, 6, 7, 8, 9, 10, 11, 12, 13, 14, 15, or all 16 interface residues in addition to residues 8-10 are identical to those in the reference polypeptide.

Para. 5. The polypeptide of any one of Paras. 1-4, wherein at least 1, 2, 3, 4, 5, 6, 7, or all 8 core residues are identical to those in the reference polypeptide.

Para. 6. The polypeptide of any one of Paras. 1-5, wherein substitutions relative to the reference polypeptide are selected from those listed in Table 2, Option 1.

Para. 7. The polypeptide of any one of Paras. 1-5, wherein substitutions relative to the reference polypeptide are selected from those listed in Table 2, Option 2.

Para. 8. The polypeptide of any one of Paras. 1-7, wherein substitutions relative to the reference polypeptide are conservative amino acid substitutions.

Para. 9. The polypeptide of any one of Paras. 1-8, wherein, relative to the reference polypeptide, 1, 2, 3, 4, 5, 6, 7, 8, 9, 10, 11, 12, or all 13 of the following are true:
Residue 3 is L or V;
Residue 5 is I or V;
Residue 6 is H;
Residue 13 is S or R;
Residue 14 is S;
Residue 31 is E or R;
Residue 32 is V or R;
Residue 33 is D, K, or N;
Residue 34 is H;
Residue 35 is K or R;
Residue 58 is G;
Residue 59 is I, V, or L; and/or
Residue 60 is W.

Para. 10. The polypeptide of any one of Paras. 1-8, wherein, relative to the reference polypeptide, 1, 2, 3, 4, 5, 6, 7, or all 8 of the following are true:
Residue 31 is E or R;
Residue 32 is V or R;
Residue 33 is D, K, or N;
Residue 34 is H;
Residue 35 is K or R;
Residue 58 is G;
Residue 59 is I, V, or L; and/or
Residue 60 is W.

Para. 11. The polypeptide of any one of Paras. 1-10, wherein, relative to the reference polypeptide, residue 58 is G.

Para. 12. The polypeptide of any one of Paras. 1-11, wherein substitutions relative to the reference polypeptide are selected from those listed in Table 2, Option 3.

Para. 13. The polypeptide of any one of Paras. 1-12, comprising an insertion in one or more loop regions of the polypeptide.

Para. 14. A fusion protein, comprising:
(a) the polypeptide of any one of Paras. 1-14; and
(b) one or more functional domains.

Para. 15. The fusion protein of Para. 14, wherein the one or more functional domains comprises a multimerization domain.

Para. 16. The polypeptide or fusion protein of any one of Paras. 1-15, wherein the polypeptide binds integrin α5β1 with an affinity of between about 0.025 nm and about 50 μm, or between about 0.05 nm and about 8 μm, or between about 0.05 nm and about 8 μm, or between about 0.05 nm and about 2.5 μm, or between about 0.05 nm and about 700 nm, or between about 0.05 nm and about 0.5 nm.

Para. 17. A nucleic acid encoding the polypeptide or fusion protein of any one of Paras. 1-16.

Para. 18. An expression vector comprising the nucleic acid of Para. 17 operatively linked to a suitable control sequence.

Para. 19. A host cell comprising the polypeptide, fusion protein, nucleic acid, or expression vector of any one of Paras. 1-18.

Para. 20. A pharmaceutical composition, comprising:
(a) the polypeptide, the nucleic acid, the expression vector, and/or the host cell of any preceding Para.; and
(b) a pharmaceutically acceptable carrier.

Para. 21. The pharmaceutical composition of Para. 20, further comprising one or more additional active agents selected from the group consisting of angiogenesis inhibitors (including but not limited to axitinib, bevacizumab, cabozantinib, everolimus, lenalidomide, lenvatinib mesylate, pazopanib, ramucirumab, regorafenib, sorafenib, sunitinib, thalidomide, vandetanib, and ziv-aflibercept), immune checkpoint inhibitors (including, but not limited to, pembrolizumab, nivolumab, and cemiplimab as anti-PD-1 antibodies, ipilimumab as an anti-CTLA-4 antibody, and atezolizumab, avelumab, and durvalumab as anti-PD-L1 antibodies), and other cancer growth inhibitors including but not limited to tyrosine kinase inhibitors (including but not limited to alectinib, brigatinib, ceritinib, crizotinib, entrectinib, lorlatinib, ALK, I, bosutinib, dasatinib, imatinib, nilotinib, ponatinib, afatinib, dacomitinib, erlotinib, gefitinib, lapatinib, neratinib, osimertinib, vandetanib, gilteritinib, midostaurin, erdafitinib, ruxolitinib, larotrectinib, axitinib, carbozantinib, lenvatinib, pazopanib, regorafenib, sorafenib, sunitinib, dabrafenib, encorafenib, vemurafenib, acalabrutinib, ibrutinib, binimetinib, cobimetinib, trametinib, abemaciclib, palbociclib, or ribociclib), proteasome inhibitors (including but not limited to ortezomib, carfizomib, ixazomib, delanzomib, oprozomib, and marizomib), mTOR inhibitors (including but not limited to everolimus, sirolimus, temsirolimus, everolimus, sirolimus, sirolimus protein-bound, and everolimus), PI3K inhibitors (including but not limited to copanlisib, alpelisib, idelalisib, duvelisib and umbralisib), histone deacetylase inhibitors (including but not limited to vorinostat, romidepsin, panobinostat, and belinostat), and Hedgehog pathway blockers (including but not limited to vismodegib, sonidegib, and glasdegib).

Para. 22. A method for treating cancer, comprising administering to a subject having cancer an amount effective to treat the cancer of the polypeptide, fusion protein, nucleic acid, expression vector, host cell, and/or pharmaceutical composition of any one of Paras. 1-21.

Para. 23. A method for inhibiting tumor metastasis, comprising administering to a subject having cancer an amount effective to inhibit tumor metastasis of the polypeptide, fusion protein, nucleic acid, expression vector, host cell, and/or pharmaceutical composition of any one of Paras. 1-21.

Para. 24. A method for treating cancer, comprising administering to a subject undergoing radiation therapy to treat cancer an amount effective to enhance effectiveness of the radiation therapy of the polypeptide, fusion protein, nucleic acid, expression vector, host cell, and/or pharmaceutical composition of any one of Paras. 1-21.

Para. 25. The method of Para. 24, wherein the radiation therapy comprises external beam radiation therapy.

Para. 26. The method of Para. 24, wherein the radiation therapy comprises targeted radiotherapy.

Para. 27. The method of any one of Paras. 22-26, wherein the cancer is selected from the group consisting of glioblastoma, glioma, colon cancer, colorectal cancer, lung cancer, breast cancer, prostate cancer, ovarian cancer, cervical cancer, Ewing sarcoma, acute lymphoblastic leukemia, melanoma, basal cell carcinoma, multiple myeloma, osteosarcoma, squamous carcinoma, head and neck squamous cell carcinoma, mesothelioma, pancreatic ductal adenocarcinoma, gastric cancer, cholangiocarcinoma, epidermoid carcinoma, chondrosarcoma, neuroblastoma, rectal cancer, and transitional carcinoma.

Para. 28. A method for inhibiting cell migration and/or attachment, comprising administering to a subject in need thereof an amount effective to inhibit cell migration and/or attachment of the polypeptide, fusion protein, nucleic acid, expression vector, host cell, and/or pharmaceutical composition of any one of Paras. 1-21.

Para. 29. The method of Para. 28, wherein the method inhibits human umbilical vein endothelial cell (HUVEC) cell migration and/or attachment.

Para. 30. A method for treating a vascular disease, comprising administering to a subject having a vascular disease an amount effective to treat the vascular disease of the polypeptide, fusion protein, nucleic acid, expression vector, host cell, and/or pharmaceutical composition of any one of Paras. 1-21.

Para. 31. The method of Para. 30, wherein the vascular disease is selected from the group consisting of vascular diabetic retinopathy, choroidal neovascularization, stroke, and pulmonary arterial hypertension.

Para. 32. A method for treating rheumatoid arthritis, comprising administering to a subject having rheumatoid arthritis an amount effective to treat the rheumatoid arthritis of the polypeptide, fusion protein, nucleic acid, expression vector, host cell, and/or pharmaceutical composition of any one of Paras. 1-21.

Para. 33. A method for treating angiogenesis, comprising administering to a subject having a disorder associated with pathogenic angiogenesis an amount effective to limit the pathogenic angiogenesis of the polypeptide, fusion protein, nucleic acid, expression vector, host cell, and/or pharmaceutical composition of any one of Paras. 1-21.

Para. 34. The method of Para. 33, wherein the disorder associated with pathogenic angiogenesis is selected from the group consisting of macular degeneration (such as wet age-related macular degeneration), diabetic retinopathy, rheumatoid arthritis, atherosclerosis, and cardiovascular diseases.

Para. 35. The method of any one of Paras. 22-34, further comprising administering to the subject one or more additional active agents selected from the group consisting of angiogenesis inhibitors (including but not limited to axitinib, bevacizumab, cabozantinib, everolimus, lenalidomide, lenvatinib mesylate, pazopanib, ramucirumab, regorafenib, sorafenib, sunitinib, thalidomide, vandetanib, and ziv-aflibercept), immune checkpoint inhibitors (including, but not limited to, pembrolizumab, nivolumab, and cemiplimab as anti-PD-1 antibodies, ipilimumab as an anti-CTLA-4 antibody, and atezolizumab, avelumab, and durvalumab as anti-PD-L1 antibodies), and other cancer growth inhibitors including but not limited to tyrosine kinase inhibitors (including but not limited to alectinib, brigatinib, ceritinib, crizotinib, entrectinib, lorlatinib, ALK, I, bosutinib, dasatinib, imatinib, nilotinib, ponatinib, afatinib, dacomitinib, erlotinib, gefitinib, lapatinib, neratinib, osimertinib, vandetanib, gilteritinib, midostaurin, erdafitinib, ruxolitinib, larotrectinib, axitinib, carbozantinib, lenvatinib, pazopanib, regorafenib, sorafenib, sunitinib, dabrafenib, encorafenib, vemurafenib, acalabrutinib, ibrutinib, binimetinib, cobimetinib, trametinib, abemaciclib, palbociclib, or ribociclib), proteasome inhibitors (including but not limited to ortezomib, carfizomib, ixazomib, delanzomib, oprozomib, and marizomib), mTOR inhibitors (including but not limited to everolimus, sirolimus, temsirolimus, everolimus, sirolimus, sirolimus protein-bound, and everolimus), PI3K inhibitors (including but not limited to copanlisib, alpelisib, idelalisib, duvelisib and umbralisib), histone deacetylase inhibitors (including but not limited to vorinostat, romidepsin, panobinostat, and belinostat), and Hedgehog pathway blockers (including but not limited to vismodegib, sonidegib, and glasdegib)

Para. 36. A medical device or implant comprising a plurality of the polypeptide, fusion protein, or pharmaceutical composition of any one of Paras. 1-21 coated on or in the medical device or implant.

Para. 37. Use of the polypeptide, fusion protein, nucleic acid, expression vector, host cell, and/or pharmaceutical composition of any one of Paras. 1-21 for treating cancer in a subject.

Para. 38. Use of the polypeptide, fusion protein, nucleic acid, expression vector, host cell, and/or pharmaceutical composition of any one of Paras. 1-21 for inhibiting tumor metastasis in a subject.

Para. 39. Use of the polypeptide, fusion protein, nucleic acid, expression vector, host cell, and/or pharmaceutical composition of any one of Paras. 1-21 to enhance the effectiveness of radiation therapy in a subject undergoing radiation therapy to treat cancer.

Para. 40. Use of the polypeptide, fusion protein, nucleic acid, expression vector, host cell, and/or pharmaceutical composition of any one of Paras. 1-21 for treating cancer in a subject undergoing radiation therapy to treat cancer, wherein the polypeptide, fusion protein, nucleic acid, expression vector, host cell, and/or pharmaceutical composition enhances the effectiveness of the radiation therapy.

Para. 41. The use of Para. 39 or 40, wherein the radiation therapy comprises external beam radiation therapy.

Para. 42. The use of Para. 39 or 40, wherein the radiation therapy comprises targeted radiotherapy.

Para. 43. The use of any one of Paras. 37-42, wherein the cancer is selected from the group consisting of glioblastoma, glioma, colon cancer, colorectal cancer, lung cancer, breast cancer, prostate cancer, ovarian cancer, cervical cancer, Ewing sarcoma, acute lymphoblastic leukemia, melanoma, basal cell carcinoma, multiple myeloma, osteosarcoma, squamous carcinoma, head and neck squamous cell carcinoma, mesothelioma, pancreatic ductal adenocarcinoma, gastric cancer, cholangiocarcinoma, epidermoid carcinoma, chondrosarcoma, neuroblastoma, rectal cancer, and transitional carcinoma.

Para. 44. Use of the polypeptide, fusion protein, nucleic acid, expression vector, host cell, and/or pharmaceutical composition of any one of Paras. 1-21 for inhibiting cell migration and/or attachment in a subject.

Para. 45. The use of Para. 44, wherein the use inhibits human umbilical vein endothelial cell (HUVEC) cell migration and/or attachment.

Para. 46. Use of the polypeptide, fusion protein, nucleic acid, expression vector, host cell, and/or pharmaceutical composition of any one of Paras. 1-21 for treating a vascular disease in a subject.

Para. 47. The use of Para. 30, wherein the vascular disease is selected from the group consisting of vascular diabetic retinopathy, choroidal neovascularization, stroke, and pulmonary arterial hypertension.

Para. 48. Use of the polypeptide, fusion protein, nucleic acid, expression vector, host cell, and/or pharmaceutical composition of any one of Paras. 1-21 for treating rheumatoid arthritis in a subject.

Para. 49. Use of the polypeptide, fusion protein, nucleic acid, expression vector, host cell, and/or pharmaceutical composition of any one of Paras. 1-21 for treating angiogenesis in a subject having a disorder associated with pathogenic angiogenesis.

Para. 50. The use of Para. 49, wherein the disorder associated with pathogenic angiogenesis is selected from the group consisting of macular degeneration (such as wet age-related macular degeneration), diabetic retinopathy, rheumatoid arthritis, atherosclerosis, and cardiovascular diseases.

### References

pubmed.ncbi.nlm.nih.gov/31913286/#:˜:text=Integrin%2Dspecific%20hydrogels%20regulate%20hMSC,repair%20compared%20to%20other%20peptides.

Hou J. Yan D, Liu Y, Huang P. Cui H. The Roles of Integrin α5β1 in Human Cancer. Onco Targets Ther. 2020 Dec. 31: 13:13329-13344. doi: 10.2147/OTT.S273803. PMID: 33408483; PMCID: PMC7781020.

1. A polypeptide comprising or consisting of an amino acid sequence at least 50%, 55%, 60%, 70%, 75%, 80%, 85%, 90%, 91%, 92%, 93%, 94%, 95%, 96%, 97%, 98%, 99%, or 100% identical to the amino acid sequence selected from SEQ ID NO:1-14, not including any insertions, wherein residues 8-10 relative to the reference polypeptide are RGD, and wherein the polypeptide selectively binds to α(5)β(1) integrin.

2.-3. (canceled)

4. The polypeptide of claim 1, wherein at least 1, 2, 3, 4, 5, 6, 7, 8, 9, 10, 11, 12, 13, 14, 15, or all 16 interface residues in addition to residues 8-10 are identical to those in the reference polypeptide.

5. The polypeptide of claim 1, wherein at least 1, 2, 3, 4, 5, 6, 7, or all 8 core residues are identical to those in the reference polypeptide.

6. The polypeptide of claim 1, wherein substitutions relative to the reference polypeptide are selected from those listed in Table 2, Option 1, or are selected from those listed in Table 2, Option 2, or are selected from those listed in Table 2, Option 3.

7. (canceled)

8. The polypeptide of claim 1, wherein substitutions relative to the reference polypeptide are conservative amino acid substitutions.

9. The polypeptide of claim 1, wherein, relative to the reference polypeptide, 1, 2, 3, 4, 5, 6, 7, 8, 9, 10, 11, 12, or all 13 of the following are true:
Residue 3 is L or V;
Residue 5 is I or V;
Residue 6 is H;
Residue 13 is S or R;
Residue 14 is S;
Residue 31 is E or R;
Residue 32 is V or R;
Residue 33 is D, K, or N;
Residue 34 is H;
Residue 35 is K or R;
Residue 58 is G;
Residue 59 is I, V, or L; and/or
Residue 60 is W.

10. The polypeptide of claim 1, wherein, relative to the reference polypeptide, 1, 2, 3, 4, 5, 6, 7, or all 8 of the following are true:
Residue 31 is E or R;
Residue 32 is V or R;
Residue 33 is D, K, or N;
Residue 34 is H;
Residue 35 is K or R;
Residue 58 is G;
Residue 59 is I, V, or L; and/or
Residue 60 is W.

11. The polypeptide of claim 1, wherein, relative to the reference polypeptide, residue 58 is G.

12. (canceled)

13. The polypeptide of claim 1, comprising an insertion in one or more loop regions of the polypeptide.

14. A fusion protein, comprising:
(a) the polypeptide of claim 1; and
(b) one or more functional domains.

15. The fusion protein of claim 14, wherein the one or more functional domains comprises a multimerization domain.

16. The polypeptide of claim 1, wherein the polypeptide binds integrin α5β1 with an affinity of between about 0.025 nm and about 50 μm, or between about 0.05 nm and about 8 μm, or between about 0.05 nm and about 8 μm, or between about 0.05 nm and about 2.5 μm, or between about 0.05 nm and about 700 nm, or between about 0.05 nm and about 0.5 nm.

17. A nucleic acid encoding the polypeptide of claim 1.

18. An expression vector comprising the nucleic acid of claim 17 operatively linked to a suitable control sequence.

19. A host cell comprising the expression vector of claim 18.

20. A pharmaceutical composition, comprising:
(a) the polypeptide of claim 1; and
(b) a pharmaceutically acceptable carrier.

21. The pharmaceutical composition of claim 20, further comprising one or more additional active agents selected from the group consisting of angiogenesis inhibitors (including but not limited to axitinib, bevacizumab, cabozantinib, everolimus, lenalidomide, lenvatinib mesylate, pazopanib, ramucirumab, regorafenib, sorafenib, sunitinib, thalidomide, vandetanib, and ziv-aflibercept), immune checkpoint inhibitors (including, but not limited to, pembrolizumab, nivolumab, and cemiplimab as anti-PD-1 antibodies, ipilimumab as an anti-CTLA-4 antibody, and atezolizumab, avelumab, and durvalumab as anti-PD-L1 antibodies), and other cancer growth inhibitors including but not limited to tyrosine kinase inhibitors (including but not limited to alectinib, brigatinib, ceritinib, crizotinib, entrectinib, lorlatinib, ALK, I, bosutinib, dasatinib, imatinib, nilotinib, ponatinib, afatinib, dacomitinib, erlotinib, gefitinib, lapatinib, neratinib, osimertinib, vandetanib, gilteritinib, midostaurin, erdafitinib, ruxolitinib, larotrectinib, axitinib, carbozantinib, lenvatinib, pazopanib, regorafenib, sorafenib, sunitinib, dabrafenib, encorafenib, vemurafenib, acalabrutinib, ibrutinib, binimetinib, cobimetinib, trametinib, abemaciclib, palbociclib, or ribociclib), proteasome inhibitors (including but not limited to ortezomib, carfizomib, ixazomib, delanzomib, oprozomib, and marizomib), mTOR inhibitors (including but not limited to everolimus, sirolimus, temsirolimus, everolimus, sirolimus, sirolimus protein-bound, and everolimus), PI3K inhibitors (including but not limited to copanlisib, alpelisib, idelalisib, duvelisib and umbralisib), histone deacetylase inhibitors (including but not limited to vorinostat, romidepsin, panobinostat, and belinostat), and Hedgehog pathway blockers (including but not limited to vismodegib, sonidegib, and glasdegib).

22.-23. (canceled)

24. A method for treating cancer, comprising administering to a subject undergoing radiation therapy to treat cancer an amount effective to enhance effectiveness of the radiation therapy of the polypeptide of claim 1.

25.-27. (canceled)

28. A method for inhibiting cell migration and/or attachment, comprising administering to a subject in need thereof an amount effective to inhibit cell migration and/or attachment of the polypeptide of claim 1.

29. (canceled)

30. A method for treating a vascular disease, comprising administering to a subject having a vascular disease an amount effective to treat the vascular disease of the polypeptide of claim 1.

31.-50. (canceled)
